(19) United States
(12) Patent Application Publication
(10) Pub. No.: US 2026/0364135 A1
(43) Pub. Date: Nov. 19, 2026

(54) Compositions and Methods for Treating Steroid Hormone-Related Diseases or Disorders

(71) Applicant: Yale University, New Haven, CT (US)

(72) Inventors: YungChi Cheng, New Haven, CT (US); Wing Lam, New Haven, CT (US); Zaoli Jiang, New Haven, CT (US)

(21) Appl. No.: 19/388,180

(22) Filed: Nov. 13, 2025

Related U.S. Application Data
(63) Continuation of application No. 17/057,999, filed on May 30, 2019, now abandoned.
(60) Provisional application No. 62/679,386, filed on Jun. 1, 2018.

(51) Int. Cl. A61K 36/74; A61K 9/00; A61K 31/704; A61K 45/06; A61P 35/00

(52) U.S. Cl. CPC A61K 36/74; A61K 9/0019; A61K 31/704; A61K 45/06; A61P 35/00

(57) ABSTRACT

The invention relates in one aspect to the unexpected discovery that herbal extracts of the Rubia cordifolia plant are potent inhibitors of a range of receptors, proteins and enzymes implicated in the pathology of a number of common diseases and disorders. In certain embodiments, the method is useful for treating at least one disease or disorder related to a steroid hormone receptor including prostate cancer, breast cancer, ovarian cancer, lung cancer, leukemia and lymphoma. In other embodiments, the method is useful for treating at least one disease or disorder related to the expression of at least one protein selected from the group consisting of Brd4, Brd2, cyclin D1, p53, Gata3 and CD47.

### CROSS-REFERENCE TO RELATED APPLICATIONS

The present application is a continuation of, and claiming priority to U.S. Non-Provisional patent application Ser. No. 17/057,999 filed on Nov. 23, 2020, which is a 35 U.S.C. § 371 national phase application from, and claiming priority to, International Application No. PCT/US2019/034548, filed May 30, 2019, which claims priority under 35 U.S.C. § 119(e) to U.S. Provisional Patent Application No. 62/679,386, filed Jun. 1, 2018, which are hereby incorporated by reference in their entireties herein.

### REFERENCE TO SEQUENCE LISTING

The Sequence Listing concurrently submitted herewith as a xml file named “047162-7175xx.xml” created on Nov. 10, 2025 and having a size of 11,053 bytes is herein incorporated by reference in its entirety.

### BACKGROUND OF THE INVENTION

Progesterone, estrogen, androgen, and glucocorticoid receptors belong to a broad class of steroid hormone receptors. Steroid hormone receptors are ligand-dependent intracellular transcription factors that have been shown to influence the development and growth of a variety of cancers, including, but not limited to, prostate cancer, breast cancer, ovarian cancer, lung cancer, leukemia and lymphoma. Hormonal therapies have shown promise in treating these kinds of cancers by modulating steroid hormone receptor activity. These results suggest that modulating steroid hormone receptor activity can be a potential target in the treatment of various cancers and other hormone receptor related diseases and disorders.

There remains an unmet need in the art for novel compositions and methods for the treatment of steroid hormone receptor related diseases and/or disorders. The present invention satisfies this unmet need.

### BRIEF SUMMARY OF THE INVENTION

The invention provides a method of treating at least one disease or disorder in a subject. In certain embodiments, the method comprises administering to the subject a therapeutically effective amount of an herbal extract of Rubia cordifolia or a fraction thereof. In certain embodiments, the method comprises administering to the subject any active chemical species present in the herbal extract of Rubia cordifolia or the fraction thereof.

In certain embodiments, the at least one disease or disorder is related to the activity of at least one steroid hormone receptor or the expression of at least one protein selected from the group consisting of Brd4, Brd2, cyclin D1, p53, Gata3, Poly[ADP-ribose]polymerase-1 (PARP-1), and CD47.

In certain embodiments, the active chemical species present in the herbal extract is selected from the group consisting of 1,3,6-Trihydroxy-2-methyl-9,10-anthracenedione 3-O-[α-L-Rhamnopyranosyl-(1→2)-6-O-acetyl-β-D-glucopyranoside](RGA), 3,6-Trihydroxy-2-methyl-9,10-anthracenedione 3-O-[α-L-Rhamnopyranosyl-(1→2)-β-D-glucopyranoside](RG) and salts, solvates, isomers, tautomers or prodrugs thereof

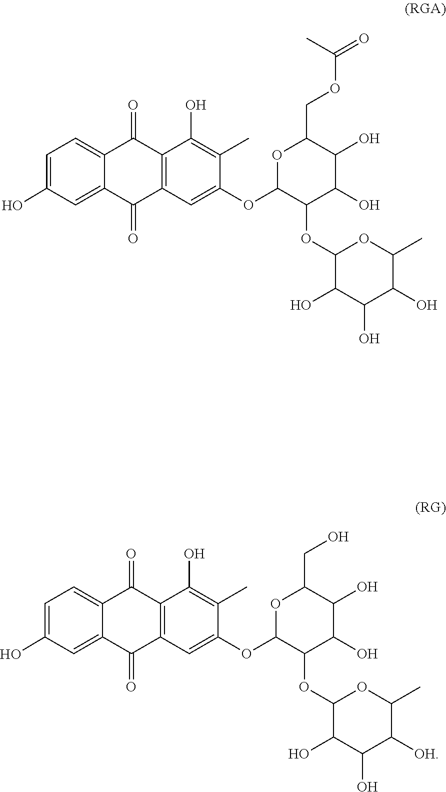 [SMILES: CC(=O)OCC1OC(Oc2cc3c(c(O)c2C)C(=O)c2ccc(O)cc2C3=O)C(OC2OC(C)C(O)C(O)C2O)C(O)C1O ; Cc1c(OC2OC(CO)C(O)C(O)C2OC2OC(C)C(O)C(O)C2O)cc2c(c1O)C(=O)c1ccc(O)cc1C2=O]

In certain embodiments, the herbal extract or the fraction thereof comprises RGA or salts, solvates, isomers, tautomers or prodrugs thereof.

In certain embodiments, the herbal extract or the fraction thereof further comprises RG or salts, solvates, isomers, tautomers or prodrugs thereof.

In certain embodiments, the at least one steroid hormone receptor is at least one selected from the group consisting of progesterone receptors, estrogen receptors, androgen receptors, and glucocorticoid receptors.

In certain embodiments, the at least one disease or disorder related to the activity of at least one steroid hormone receptor is a cancer selected from the group consisting of prostate cancer, breast cancer, ovarian cancer, lung cancer, uterine cancer, pancreatic cancer, colon cancer, hepatocellular carcinoma, glioblastoma, multiple myeloma, NUT carcinoma, leukemia, and lymphoma.

In certain embodiments, the cancer is a treatment resistant cancer selected from the group consisting of castration resistant prostate cancer, enzalutamide resistant prostate cancer, glucocorticoid receptor mediated resistant prostate cancer, BRCA1 (double strand break repair) deficiency cancer, double negative breast cancer, and triple negative breast cancer.

In certain embodiments, the at least one disease or disorder related to the activity of at least one steroid hormone receptor is selected from the group consisting of prostate hyperplasia, Cushing's syndrome, androgenetic alopecia, acne, seborrhea, hirsutism (excessive body hair), hidradenitis suppurativa, sexual dysfunction, precocious puberty (in both males and females), polycystic ovary syndrome, mastodynia (breast pain/tenderness), breast fibroids, mammoplasia (breast enlargement), macromastia (breast hypertrophy), gynecomastia, melasma, menorrhagia, endometriosis, endometrial hyperplasia, adenomyosis, uterine fibroids, and posttraumatic stress disorder (PTSD).

In certain embodiments, the at least one disease or disorder related to expression of the at least one protein is selected from the group consisting of Huntington's disease, schizophrenia, psoriasis, mantle cell lymphoma, breast carcinoma, bladder cancer, pituitary adenomas, parathyroid adenoma, pancreatic carcinoma, head and neck squamous cell carcinomas, and non-small cell lung cancers.

In certain embodiments, the method downregulates the expression of at least one protein selected from the group consisting of Brd4, Brd2, cyclin D1, p53, Gata3, Poly[ADP-ribose]polymerase-1 (PARP-1), and CD47.

In certain embodiments, the administration inhibits hyperacetylation of histone H3 in the subject.

In certain embodiments, the administration promotes the phagocytosis of cancerous tumor cells in the subject.

In certain embodiments, the administration inhibits indoleamine-pyrrole 2,3-dioxygenase (IDO) activity in the subject.

In certain embodiments, the subject is further administered at least one immune checkpoint inhibitor. In certain embodiments, the at least one immune checkpoint inhibitor is selected from the group consisting of an anti-PD1, an anti-PD-L1, and an anti-CTLA4. In certain embodiments, the at least one immune checkpoint inhibitor is selected from the group consisting of Ipilimumab, Avelumab, Pembrolizumab, Nivolumab, Durvalumab, and Atezolizumab.

In certain embodiments, the subject is a mammal.

In certain embodiments, the subject is human.

### BRIEF DESCRIPTION OF THE DRAWINGS

For the purpose of illustrating the invention, depicted in the drawings are certain embodiments of the invention. However, the invention is not limited to the precise arrangements and instrumentalities of the embodiments depicted in the drawings.

FIG. 1 is a graph showing the effects of certain herbal water-extract preparations of Rubia cordifolia (Y9, Y1830, Y1831) on dihydrotestosterone (DHT) induced androgen receptor mediated transcriptional activity in 22RV1 prostate cancer cells. 22RV1 cells transfected with prostate-specific antigen (PSA) luciferase reporter were treated with certain batches of Rubia cordifolia water extracts for 24 h before luciferase activity was measured.

FIGS. 2A-2B are a set of graphs showing anti-androgen receptor (AR) activity for certain ethanol extracted fractions of Rubia cordifolia extracts (FIG. 2A: Y1830; FIG. 2B: Y9) after passing through a solid phase silica extraction (DSC18) column. The anti-AR activity was assessed using a 22RV1 PSA-luciferase reporter assay.

FIG. 2C is an LC-MS spectrum (−ve scanning mode) showing 2 peaks from a 50% ethanol fraction, assigned as 1,3,6-Trihydroxy-2-methyl-9,10-anthracenedione 3-O-[α-L-Rhamnopyranosyl-(1→2)-β-D-glucopyranoside](RG) with MW 578.4, and 1,3,6-Trihydroxy-2-methyl-9,10-anthracenedione 3-O-[α-L-Rhamnopyranosyl-(1→2)-6-O-acetyl-β-D-glucopyranoside](RGA) with MW 620.4. The separated anthraquinone moiety of RGA and RG was observed in a 75% ethanol fraction and was assigned as 1,3,6-Trihydroxy-2-methyl-9,10-anthracenedione (TMT) with MW 270.2.

FIG. 2D is an LC-MS spectrum (−ve scanning mode) for RGA with or without NaOH treatment. The LC-MS spectrum shows that RGA hydrolyzes to RG upon treatment with base, removing the acetyl moiety.

FIG. 3 is an LC-MS spectrum (+ve scanning mode) showing that the fraction comprising RGA may contain additional molecules with M/Z=757.4 (M+H), M/Z=332, M/Z=495.

FIGS. 4A-4B are graphs showing the effect of Y9, Y1830, RG (or the fraction comprising RG), RGA (or the fraction comprising RGA), and TMT (or the fraction comprising TMT), at equivalent doses, on the AR mediated transcriptional response of 22RV1 AR-luciferase reporter cells in the present of dihydrotestosterone (DHT).

FIGS. 4C-4D are graphs showing the effect of Y1830, RG (or the fraction comprising RG) and RGA (or the fraction comprising RGA) on the mRNA of PSA (FIG. 4C) and KLK2 (FIG. 4D). 22RV1 cells were treated with Y1830, RGA or RG overnight and mRNA was extracted for qRT-PCR to determine the mRNA expression level of PSA and KLK2 as normalized to beta-actin.

FIGS. 5A-5G are an image and graphs showing the effect of Y1830, RGA (or the fraction comprising RGA), and RG (or the fraction comprising RG) on the dexamethasone (DEX) induced glucocorticoid receptor (GR) mediated transcriptional response of 22RV1 and PC3 PSA-luciferase reporter cells. FIG. 5A is a Western blot analysis for AR and GR protein expression in LNCaP, 22RV1 and PC3 cells. FIGS. 5B-5C are graphs showing luciferase activity in response to certain doses of DEX in 22RV1 or PC3 AR-luciferase reporter cells.

FIGS. 5D-5E are graphs showing the effect of Y1830, RG (or the fraction comprising RG), and RGA (or the fraction comprising RGA) on the DEX induced glucocorticoid receptor mediated transcriptional response of 22RV1 and PC3 PSA-luciferase reporter cells. FIGS. 5F-5G are graphs showing the effect of Y1830 on SGK1 (target gene of GR) mRNA expression with or without DEX using qRT-PCR.

FIGS. 6A-6D are an image and graphs showing the effect of Y1830, RGA (or the fraction comprising RGA), and RG (or the fraction comprising RG) on the AR or GR driven luciferase activity of standard LNCaP cells or GR overexpressing LNCaP cells. FIG. 6A is a Western blot analysis for AR and GR protein expression in LNCaP and GR overexpressing LNCaP cells. FIG. 6B is a graph showing the response of LNCaP and GR overexpressing LNCaP cells to DHT and DEX. FIG. 6C is a set of graphs showing the effect of Y1830, RGA (or the fraction comprising RGA), RG (or the fraction comprising RG) and Enzalutamide (ENZA) on AR driven luciferase activity in LNCaP cells. FIG. 6D is a set of graphs showing the effect of Y1830, RGA (RGA containing fraction), RG (or the fraction comprising RG) and ENZA on AR (stimulated with DHT) or GR (stimulated with DEX) driven luciferase activity in GR overexpressing LNCaP cells. Both LNCaP and GR overexpressing LNCaP cells were carrying luciferase reporters with AR response elements.

FIG. 7A-7E are graphs showing the growth of certain prostate cell lines in present or absence of DHT or DEX for 4 days.

FIGS. 8A-8B are Western blot analyses showing the effect of Y1830, RGA (or the fraction comprising RGA) and RG (or the fraction comprising RG) on protein expression for GR, AR, truncated AR (AR-V), beta-catenin (b-cat) and cyclin D1 (cycD1) in the presence or absence of DHT and MG132 in 22RV1 cells. 22RV1 cells were treated with Y1830, RGA (or the fraction comprising RGA) and RG (or the fraction comprising RG) in the presence or absence of DHT and MG132 for 24 h. Western blot analysis was used to determine the protein expression using protein specific antibodies.

FIGS. 9A-9B are Western blot analyses showing the effect of Y1830, RGA (or the fraction comprising RGA), and RG or the fraction comprising RG on protein expression for ERa, ERb, GR, PR-b, PRa, AR, AR-V, b-cat and cycD1 in the presence or absence of DHT and MG132 in MCF7 cells. MCF7 cells were treated with Y1830, RGA (or the fraction comprising RGA), and RG (or the fraction comprising RG) in the presence or absence of E2 and MG132 for 24 h. Western blot analysis was used to determine the protein expression using protein specific antibodies.

FIG. 10 is a Western blot analysis showing the effect of Y1830, RGA (or the fraction comprising RGA) and RG (or the fraction comprising RG) on protein expression for p53, H2AX, H2AX-ser139, PARP1, and GATA3 proteins in the presence or absence of E2 in MCF7 cells. MCF7 cells were treated with Y1830, RGA (or the fraction comprising RGA) and RG (or the fraction comprising RG) in the presence or absence of E2 for 24 h. Western blot analysis was used to determine the protein expression using protein specific antibodies.

FIGS. 11A-11B are Western blot analyses showing the effect of Y1830, RGA (or the fraction comprising RGA) and RG (or the fraction comprising RG) on the protein expression of Brd4, Brd2 and histone acetylation at lysine 9, 14 and 27 in the presence or absence of E2 in MCF7 cells (FIG. 11A) and in the presence or absence of DHT in 22R1 cells (FIG. 11B). Cells were treated with Y1830, RGA (or the fraction comprising RGA), and RG (or the fraction comprising RG) in the presence or absence of E2 or DHT for 24 h. Western blot analysis was used to determine the protein expression using protein specific antibodies. β-actin or histone 3 was used to normalize protein loading.

FIGS. 12A-12C are graphs showing the effect of Y1830, RGA (or the fraction comprising RGA), and RG (or the fraction comprising RG) on the cell growth of MCF7, T4D, MDAMB453 and MDAMBA231 cells in the presence (FIG. 12C) or absence (FIG. 12B) of E2, for 4 days.

FIG. 13 is a graph showing the effect of certain preparations of Rubia cordifolia on indoleamine-pyrrole 2,3-dioxygenase (IDO) activity in an in vitro assay. 500 μg/ml of Y1830, Y9 or N9 were added to lysed HEK293 cells, which had been transfected with mouse IDO expression plasmids for 48 h, in the presence of 1 mM L-tryptophan for 90 min. The concentration of kynurenine in the culture medium was measured using a colorimetric based assay.

FIG. 14 is a set of graphs comparing the inhibition profiles of certain ethanol elutions of Y1830 from solid a phase extract C18 column on AR activity and IDO activity. The LC-MS chemical profile for the E10%, E30%, E50% and E75% (where E=Ethanol) extracts are shown on the bottom panel.

FIGS. 15A-15B are the graphs showing effects of Y1830, RGA (or the fraction comprising RGA), and RG (or the fraction comprising RG) on 22RV1 tumor growth and body-weight of nude mice during the treatment.

### DETAILED DESCRIPTION OF THE INVENTION

The invention relates in one aspect to the unexpected discovery that herbal extracts of the Rubia cordifolia plant (also known as the common madder, Indian madder, manjistha, majith, tamaralli, or manditti) are potent inhibitors of certain receptors, proteins, and enzymes implicated in the pathology of a number of common diseases and disorders.

In one aspect, the invention provides methods for treating a range of diseases and disorders using an herbal extract of Rubia cordifolia or a fraction thereof or any active chemical species present in the herbal extract or a fraction thereof. In certain embodiments, the method is useful for treating at least one disease or disorder related to a steroid hormone receptor, such as but not limited to progesterone receptors, estrogen receptors, androgen receptors and glucocorticoid receptors. In other embodiments, the method is useful for treating at least one disease or disorder related to the expression of at least one protein selected from the group consisting of Brd4, Brd2, cyclin D1, p53, Gata3 and CD47. In yet other embodiments, the herbal extracts and active chemical species isolated therefrom can be used to treat at least one cancer selected from the group consisting of prostate cancer, breast cancer, ovarian cancer, lung cancer, uterine cancer, pancreatic cancer, colon cancer, hepatocellular carcinoma, glioblastoma, multiple myeloma, NUT carcinoma, leukemia and lymphoma.

In yet other embodiments, the herbal extracts and active chemical species isolated therefrom can be used to treat disorders related to hyperacetylation of histone H3, such as but not limited to Huntington's disease and schizophrenia.

### Methods

In one aspect, the invention provides a method of treating at least one disease or disorder in a subject. In certain embodiments, the method comprises administering to the subject a therapeutically effective amount of an herbal extract of Rubia cordifolia or a fraction thereof or any active chemical species present in the herbal extract or a fraction thereof.

In certain embodiments, the active chemical species present in the herbal extract is 1,3,6-Trihydroxy-2-methyl-9,10-anthracenedione 3-O-[α-L-Rhamnopyranosyl-(1→2)-6-O-acetyl-β-D-glucopyranoside](RGA), or a salt, solvate, isomer, tautomer, or prodrug thereof.

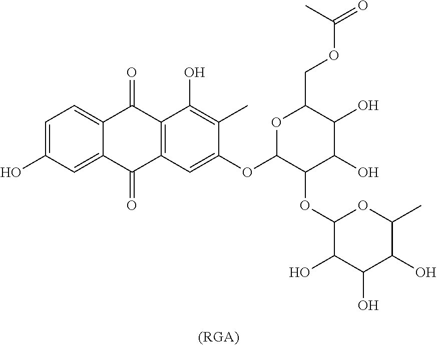 [SMILES: CC(=O)OCC1OC(Oc2cc3c(c(O)c2C)C(=O)c2ccc(O)cc2C3=O)C(OC2OC(C)C(O)C(O)C2O)C(O)C1O]

In certain embodiments, the active chemical species present in the herbal extract is 1,3,6-Trihydroxy-2-methyl-9,10-anthracenedione 3-O-[α-L-Rhamnopyranosyl-(1→2)-β-D-glucopyranoside](RG), or a salt, solvate, isomer, tautomer or prodrug thereof.

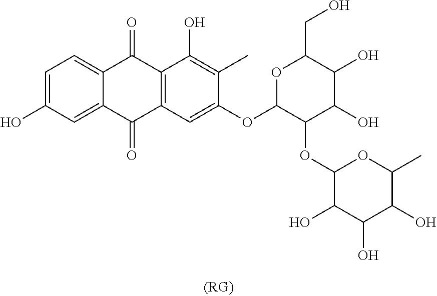 [SMILES: Cc1c(OC2OC(CO)C(O)C(O)C2OC2OC(C)C(O)C(O)C2O)cc2c(c1O)C(=O)c1ccc(O)cc1C2=O]

In certain embodiments, the method treats at least one disease or disorder related to the activity of at least one steroid hormone receptor. In other embodiments, the at least one steroid hormone receptor is selected from the group consisting of progesterone receptors, estrogen receptors, androgen receptors and glucocorticoid receptors. In yet other embodiments, the method downregulates the expression of at least one steroid hormone receptor selected from the group consisting of androgen receptors, estrogen receptor alpha, and progesterone receptors. In yet other embodiments, the method inhibits the activity of glucocorticoid receptors.

In certain embodiments, the at least one disease or disorder is a cancer. In other embodiments, the cancer is at least one selected from the group consisting of prostate cancer, breast cancer, ovarian cancer, lung cancer, uterine cancer, pancreatic cancer, colon cancer, hepatocellular carcinoma, glioblastoma, multiple myeloma, NUT carcinoma, leukemia and lymphoma.

In yet other embodiments, the cancer is a treatment resistant form of cancer. In yet other embodiments, the cancer is at least one selected from the group consisting of castration resistant prostate cancer, enzalutamide resistant prostate cancer, BRCA1 (double strand break repair) deficiency cancer and glucocorticoid receptor mediated resistant prostate cancer. In yet other embodiments, the cancer is double negative breast cancer or triple negative breast cancer. In yet other embodiments, the triple negative breast cancer is MBA-MB-231 or MBA-MB-453 breast cancer.

In certain embodiments, the at least one disease or disorder is prostate hyperplasia.

In certain embodiments, the at least one disease or disorder is a hormone function disease or disorder selected from the group consisting of Cushing's syndrome, androgenetic alopecia, acne, seborrhea, hirsutism (excessive body hair), hidradenitis suppurativa, sexual dysfunction, precocious puberty (in both males and females), polycystic ovary syndrome, mastodynia (breast pain/tenderness), breast fibroids, mammoplasia (breast enlargement), macromastia (breast hypertrophy), gynecomastia, melasma, menorrhagia, endometriosis, endometrial hyperplasia, adenomyosis, and uterine fibroids.

In certain embodiments, the method treats at least one psychiatric disorder related to the activity of at least one steroid hormone receptor. In other embodiments, the at least one psychiatric disorder is posttraumatic stress disorder (PTSD).

In certain embodiments, the method treats at least one disease or disorder related to expression of at least one protein selected from the group consisting of Brd4, Brd2, cyclin D1, p53, Gata3, Poly[ADP-ribose]polymerase-1 (PARP-1) and CD47. In other embodiments, the at least one disease or disorder related to expression of the at least one protein is selected from the group consisting of at least one variety of cancer described elsewhere herein and at least one inflammatory disease or disorder.

In certain embodiments, the method downregulates Brd4 expression in the subject, thereby inhibiting hyperacetylation of histone H3. In other embodiments, the method decreases histone 3 lysine 27 acetylation (H3 ac-lys27) but not H3 ac-lys9 or H3 ac-lys14 acetylation. In yet other embodiments, the method treats at least one disease or disorder related to hyperacetylation of histone H3 selected from, but not necessarily limited to, at least one variety of cancer described elsewhere herein, inflammation diseases and disorders, autoimmune diseases and disorders, Huntington's disease and schizophrenia.

In certain embodiments, the method downregulates CD47 expression in the subject. Without intending to be limited to any particular theory, the downregulation of CD47 expression on tumor cells can prevent inhibition of phagocytosis of the tumor cells by macrophages. In other embodiments, the method promotes phagocytosis of cancerous tumor cells in the subject.

In certain embodiments, the method downregulates cyclin D1 expression in the subject. In other embodiments, the method treats at least one disease or disorder selected from the group consisting of psoriasis, mantle cell lymphoma, breast carcinoma, bladder cancer, pituitary adenomas, parathyroid adenoma, pancreatic carcinoma, head and neck squamous cell carcinomas and non-small cell lung cancers.

In certain embodiments, the method inhibits indoleamine-pyrrole 2,3-dioxygenase (IDO) activity in the subject.

In certain embodiments, the method is useful as part of an immunotherapy based treatment regimen. In other embodiments, the method further comprises administering to the subject at least one immune checkpoint inhibitor. In yet other embodiments, the immune checkpoint inhibitor is selected from the group consisting of an anti-PD1, an anti-PD-L1, and an anti-CTLA4. In other embodiments, the immune checkpoint inhibitor is selected from the group consisting of Ipilimumab, Avelumab, Pembrolizumab, Nivolumab, Durvalumab and Atezolizumab.

In certain embodiments, the method further comprises administering to the subject at least one additional anti-cancer agent. In other embodiments, the at least one additional anti-cancer agent is a chemotherapeutic agent.

In another aspect, the invention provides methods of modulating one or more steroid hormone receptors in a subject for purposes other than treating a disease or disorder. In certain embodiments, the invention provides methods of terminating a pregnancy in a subject. In other embodiments, the invention provides a means of contraception in the subject. In yet other embodiments, the method comprises administering to the subject a therapeutically effective amount of an herbal extract of Rubia cordifolia or a fraction thereof or any active chemical species present in the herbal extract or a fraction thereof.

In certain embodiments, the subject is a mammal. In other embodiments, the subject is human.

In certain embodiments, the herbal extract is administered to the subject orally. In other embodiments, the herbal extract is administered in at least one form selected from the group consisting of a pill, tablet, capsule, soup, tea, concentrate, dragees, liquids, drops, and gelcaps.

In certain embodiments, the herbal extracts of the invention can be water extracts. The water extracts can be prepared through a method comprising at least one of the following steps: drying the herbs of the invention, grinding the dried herbs into an herb powder, adding the herb powder to an amount of water to form a mixture, heating the mixture to an elevated temperature for a period of time, allowing the mixture to cool down to room temperature, removing and removing any undissolved solids.

In certain embodiments, the herbal powder is added to the water in a ratio of 100 mg of herbs per 1 mL of water. In certain embodiments, the mixture is heated to a temperature of about 80 °C for about 1 h. In certain embodiments, the undissolved solids are removed by centrifuging the mixture to form a pellet and then decanting and collecting the water extract, leaving behind the solid pellet.

The active compounds of the invention may possess one or more stereocenters, and each stereocenter may exist independently in either the (R) or (S) configuration. In one embodiment, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

In one embodiment, the active compounds of the invention exist as tautomers. All tautomers are included within the scope of the compounds recited herein.

In one embodiment, compounds described herein are prepared as prodrugs. A “prodrug” is an agent converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 1,000 mg/kg of body weight/per day. The pharmaceutical compositions useful for practicing the invention may be administered to deliver a dose of from 1 ng/kg/day and 100 mg/kg/day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In certain embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating cancer) in the composition. In yet other embodiments, the compound of the invention is the only biologically active agent (i.e., capable of treating cancer) in therapeutically effective amounts in the composition.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds and/or compositions of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 400 mg to about 6,000 mg, about 500 mg to about 5,000 mg, about 750 mg to about 4,000 mg, about 1,000 mg to about 3,000 mg, about 1,000 mg to about 2,500 mg, about 20 mg to about 2,000 mg and any and all whole or partial increments therebetween.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-peritoneal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are soups, teas, concentrates, tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of a disease or disorder contemplated in the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intra-peritoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents with art-recognized alternatives and using no more than routine experimentation, are within the scope of the present application.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of 20% or ±10%, more preferably +5%, even more preferably +1%, and still more preferably ±0.1% from the specified value, as such variations are appropriate to perform the disclosed methods.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition can be formulated in any kind of combinations as mixtures of solids and liquids under a variety of solid, gel, and liquid formulations, and as a solution.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “extract” refers to a concentrated preparation or solution of a compound or drug derived from a naturally occurring source, such as an herb or other plant material. Extracts can be prepared by a number of processes including steeping an herb in solution or drying and grinding an herb into a powder and dissolving the powder in a solution. An extract can be further concentrated by removing a portion of the solvent after dissolving an amount of the desired compound in the solution. An extract may also be strained or centrifuged to remove any solid material from the solution.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or downregulate a protein, a gene, and an mRNA stability, expression, function and activity, e.g., antagonists.

The terms “patient,” “subject” or “individual” are used interchangeably herein, and refer to any animal, or cells thereof whether in vitro or in situ, amenable to the methods described herein. In a non-limiting embodiment, the patient, subject or individual is a human. In other embodiments, the patient is a non-human mammal including, for example, livestock and pets, such as ovine, bovine, porcine, canine, feline and murine mammals. In yet other embodiments, the patient is an avian animal or bird. Preferably, the patient, individual or subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

A “therapeutic” treatment is a treatment administered to a subject who exhibits signs of pathology, for the purpose of diminishing or eliminating those signs.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

The following abbreviations are used herein:
AR androgen receptor
AR-V truncated androgen receptor
BET Bromodomain and extra terminal
BRD bromodomain-containing protein
cycD1 cyclin D1
DEX dexamethasone
DHT dihydrotestosterone
E2 17β-Estradiol
GR glucocorticoid receptor
HPLC high performance liquid chromatography
IDO Indoleamine-pyrrole 2,3-dioxygenase
LC-MS liquid chromatography-mass spectrometry
LNCaP-GR glucocorticoid receptor overexpressing LNCaP cells
MW Molecular Weight
NMR nuclear magnetic resonance
PARP-1 Poly[ADP-ribose]polymerase-1
PSA prostate-specific antigen
qPCR quantitative polymerase chain reaction
RG 1,3,6-Trihydroxy-2-methyl-9,10-anthracenedione 3-O-[α-L-Rhamnopyranosyl-(1→2)-β-D-glucopyranoside]
RGA 1,3,6-Trihydroxy-2-methyl-9,10-anthracenedione 3-O-[α-L-Rhamnopyranosyl-(1→2)-6-O-acetyl-β-D-glucopyranoside]
SIRPa signal regulatory protein-α
ssDNA single-stranded DNA
TMT 1,3,6-Trihydroxy-2-methyl-9,10-anthracenedione

The following examples further illustrate aspects of the present invention. However, they are in no way a limitation of the teachings or disclosure of the present invention as set forth herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods

Rubia cordifolia Extract Preparation Methods

Dry Rubia cordifolia extract powder of was purchased from commercial sources: Y1830 (label as Rubiae Radix et Rhizoma) from PuraPharm China, Y9 (labeled as Rubia cordifolia) from E-Fong USA; and Y1831 (labeled as Rubia cordifolia) from E-Fong China. 100 mg of dry powder was dissolved by vortex in 1 ml HPLC grade water in a 2 ml centrifuge tube for 2 min. The herbal water mixture was then heated in a 80 °C water bath for 30 min. The herbal water mixture was centrifuged at 12,000 rpm in desktop centrifuge at room temperature for 5 minutes. The supernatant was transferred into a new 2 ml tube and used as a 100 mg/ml herbal water extract.

### PSA-Luciferase Assay Methods

### PSA Luciferase Reporter Cells

22RV1 or LNCaP prostate cancer cells were used in the screening study. 22RV1 or LNCaP cell lines were stably transfected with PSA promoter-PGL4.2 luciferase reporter. DHT (25 nM) was used to stimulate androgen receptor activity for 24 h.

### Luciferase Assay

Reporter cells were treated with herbal extracts at 30, 100, 300, and 1000 μg/ml for 24 h in a 37 °C-CO₂ incubator. DHT (25 nM) was used to stimulate androgen receptor activity. Dexamethasone (50 nM) was used to stimulate glucocorticoid receptor activity Cells were lysed using luciferase lysis buffer after which luciferase buffer with luciferin was added to generate luminescence. Luminescence was recorded using a luminescence microplate reader.

### Prostate Cancer Cell Culturing Methods

22RV1 or LNCaP prostate cancer cells were grown in corning T75 cell culture flasks in RPMI1640 medium supplemented with 5% FBS, 50 μg/ml Kanamycin in a 37 °C, 5% CO₂ incubator.

### Breast Cancer Cell Culturing Methods

MCF7, T47D, MDA-MB-453, MDA-MB-231 breast cancer cells were grown in corning T75 cell culture flask in RPMI1640 medium supplemented with 5% FBS, 50 μg/ml Kanamycin in a 37 °C, 5% CO₂ incubator.

### Protein Expression Western Blot Assay Methods

Total cell lysis was conducted using 2×SDS sample buffer (62.5 mM Tris-HCl, 2% SDS, 10% glycerol, 50 mM DTT, and 0.05% bromophenol blue). The samples were sonicated for 10 s to shear DNA. Cell nuclei were isolated using Tris buffer saline with 0.4% NP40. Cell extracts were then electrophoresed through 10% SDS-polyacrylamide gels and transferred to 0.2 μm nitrocellulose membranes (Bio-Rad Laboratories, Hercules, CA) with a Miniprotein II transferring apparatus (Bio-Rad). The membranes were blocked and probed in TBS-T buffer (1×TBS buffer, 0.2% Tween 20) containing 5% non-fat milk. Monoclonal rabbit anti-AR (1:5000), was used to detect androgen receptor (Abcam #133273), Glucocorticoid Receptor (D8H2) XP® Rabbit mAb #3660, ERα Antibody (F-10): sc-8002, BRD4 (E2A7X) Rabbit mAb #13440, Brd2 (D89B4) Rabbit mAb #5848, PARP (46D11) Rabbit mAb #9532, Acetyl-Histone H3 (Lys27) (D5E4) XP® Rabbit mAb #8173 and a monoclonal actin antibody diluted 1:2500 (Sigma, St. Louis, MO) was used to detect β-actin as the internal control to confirm equal protein loading. The membranes were then incubated with horseradish peroxidase-conjugated anti-mouse IgG and anti-rabbit IgG (1:5,000; Sigma). Enhanced chemiluminescence reagents (Perkin-Elmer Life Science Products, Boston, MA) were used to visualize the immunoreactive bands and the densities of protein bands were scanned analyzed using ImageJ software from the NIH.

### Cell Growth Assay Methods

20,000 cells in 1 ml of medium (phenol red free RPMI1640, 5% charcoal-dialysis FBS, 50 μg/ml Kanamyacin) were seeded per well of a 24-well plate and incubated in a 37 °C incubator with 5% CO₂ overnight. Different concentrations of herbal extract were added to each well in order to have final concentrations from 50 μg/ml to 500 μg/ml. For prostate cancer cells, DHT (25 nM) was used to stimulate androgen receptor activity. For glucocorticoid receptor overexpressed LNCaP cells, dexamethasone (50 nM) was used to stimulate glucocorticoid receptor activity. For all breast cancer cells, E2 (10 nM) was used to stimulate ER activity. Control medium without any steroid hormone was used as a control setting. After 4 days of incubation, cells were fixed and stained for 2 h with 0.5% methylene blue in 50% ethanol, followed by washing with tap water to remove unbound dye. Plates were air dried and then cells were dissolved in 1% sarkosyl (sodium lauroyl sarcosinate) by shaking at room temperature for 3 h. Cell growth was quantified based on the amount of methylene blue adsorbed by the cells as measured by a spectrophotometer (Molecular Devices) at 595 nm. All experiments were performed in triplicate wells and were repeated at least three times.

Real Time Quantitative PCR (RT-qPCR) of NRF2 and downstream genes

RNA was extracted from herb treated cells using the Roche High Pure RNA isolation kit. cDNA was then generated from RNA samples using a Bio-rad iScript Advanced cDNA synthesis kit for RT-qPCR. qPCR was performed using human NRF2, HO1, NQO1 and β-actin primers (Table 1) and iTaq™ Universal SYBR® Green Supermix in a CFX PCR machine (Bio-rad). Relative mRNA expression was calculated based on the change of the threshold cycle relative to the internal control, β-actin, using a standard curve generated by purified PCR products.

TABLE 1

| | | | |
|---|---|---|---|
| Human | F1 | CCTGGCTTCCGCAACTTACAC | SEQ ID NO. 1 |
| AR | R1 | GGACTTGTGCATGCGGTACTCA | SEQ ID NO. 2 |
| Human | F1 | GGTGGCTGTGTACAGTCATGGAT | SEQ ID NO. 3 |
| KLK2 | R1 | TGTCTTCAGGCTCAAACAGGTTG | SEQ ID NO. 4 |
| Human | F1 | ACCAGAGGAGTTCTTGACCCCAAA | SEQ ID NO. 5 |
| PSA | R1 | CCCCAGAATCACCCGAGCAG | SEQ ID NO. 6 |
| Human | F1 | GCCACGGCTGCTTCCAGCTCC | SEQ ID NO. 7 |
| β-actin | R1 | TTGTGCTGGGTGCCAGGGCAGTGA | SEQ ID NO. 8 |

### IDO Activity Assay Methods

2×10⁶ HEK293 cells were transfected with mouse IDO (2 μg/10 cm plate) for 48 h. For one plate, 1 ml PBS was used to collect cells into a 2 ml tube. Cells were centrifuged at 3,500 rpm 1 min. Cells were then sonicated in ice cold PB buffer (1 ml pH 6.5). Cell lysis was clarified by centrifuging at 12,000 rpm for 5 min at 4 °C. 25 μl cell lysis solution was mixed with herbal extract (25 μl) at desired concentrations. Reaction buffer containing 50 μl PB buffer (100 mM, pH 6.5), 10 μl methylene blue (2.5%), 100 μl catalase (20 mg/ml), 250 μl L-tryptophan (500 mM) and, for every 10 ml of total solution, 70 mg of vitamin C. The reaction buffer was then added to the cell lysis solution. The solution was allowed to react for 1.5 h at 37 °C. Trichloroacetic acid 30% (25 μl) was added and incubated at 50 °C for 1 hr. Ehrlich's reagent 0.8% [4-(Dimethylamino)benzaldehyde, 80 mg/10 ml in acetic acid, 100 μl, from Sigma Aldrich] was added. Absorbance at 540 nm was measured using a UV-vis spectrometer to determine kynurenine concentration. Absorbance at 540 nm—has been found to have a positive correlation to the amount of kynurenine in a sample.

### Inhibition of 22RV1 Prostate Cancer Growth in Nude Mice

22RV1 prostate cancer cells 5×10⁶ cells in 100 ul Matrigel were injected subcutaneously into 10 week old male NCR nude mice. When 22RV1 tumor reached 5 mm×5 mm, mice were orally fed with Y1830 (water extract, 500 mg/kg, PO, B.I.D, N5) and RGA (or the fraction comprising RGA) (equivalent dose to Y1830: 2.2 mg/kg, PO, B.I.D.) for 11 days. Tumor volume was estimated by using the formula length×width²/2.

### Example 1: Herb Extract Screening and Identification of Potential Active Compounds

Rubia cordifolia is an herb used in the traditional medicine of China and India, locally referred to as Manjistha. Traditional medicine claims that the herb can be used to “clean and cool the blood,” remove excess heat and natural toxins from the blood, support liver and kidney function, promote healthy menstruation, reduce excess water from body, reduce fever and arthritis, and support the healthy flow of blood and urine.

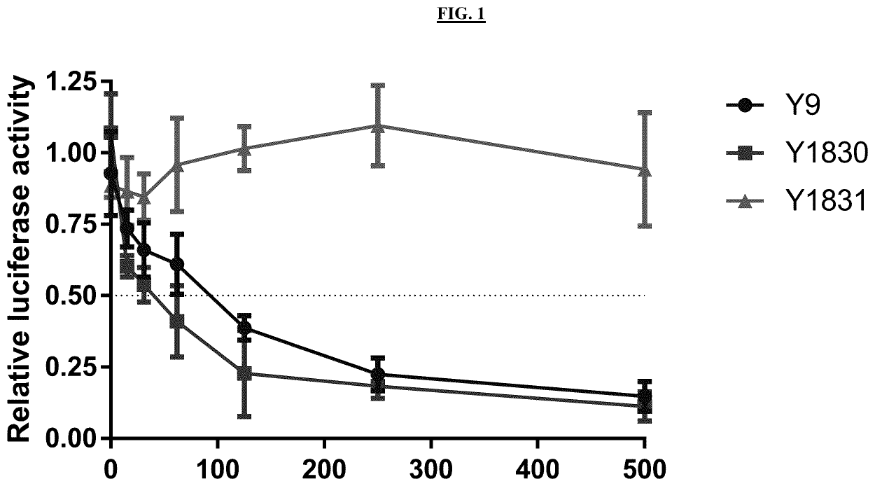

Water extracts of Rubia cordifolia were prepared from Y1830 (labeled as Rubiae Radix et Rhizoma, derived from the dried root and rhizome of the Rubia cordifolia plant, from PuraPharm China) and Y9 (Rubia cordifolia, from E-Fong USA). The prepared extracts showed an inhibitory effect on dihydrotestosterone (DHT) induced androgen receptor (AR) mediated transcriptional activity, as determined by using a luciferase reporter assay in 22RV1 prostate cancer cells. A third water extract prepared from Y1831 (Rubia cordifolia, from E-Fong China) didn't demonstrate any observable activity in inhibiting AR activity (FIG. 1).

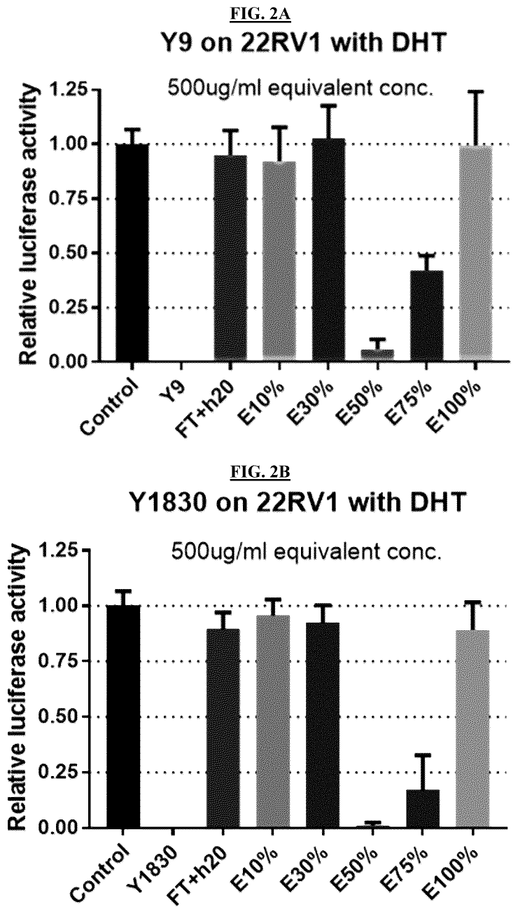

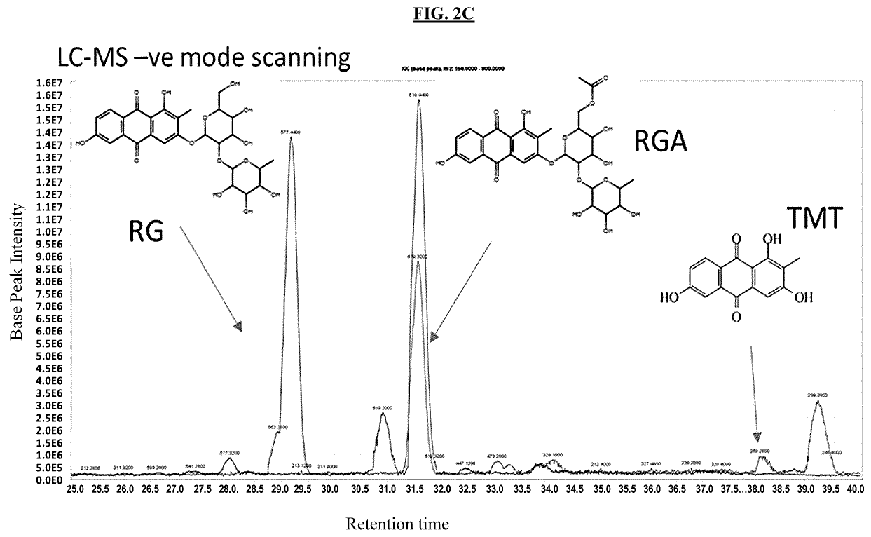

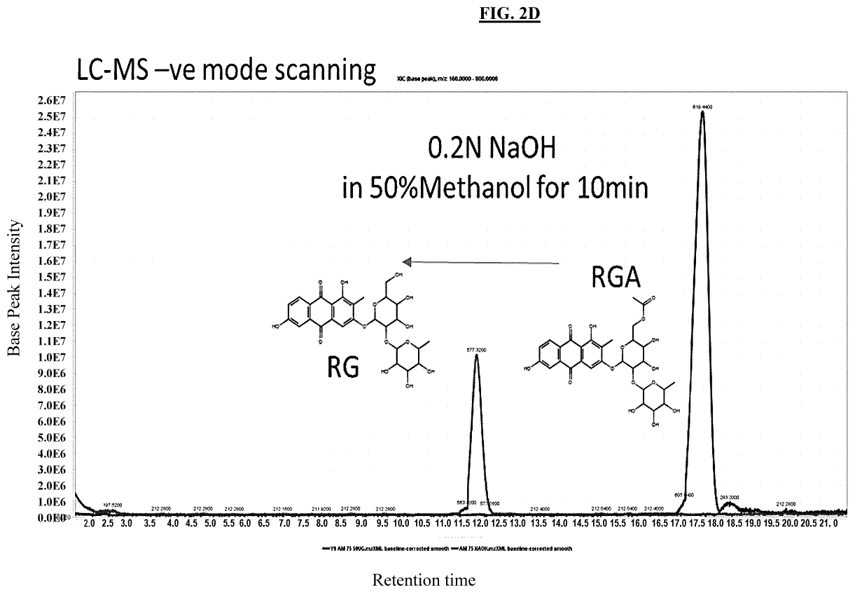

Y9 and Y1830 (100 mg/ml) water extracts were passed through a solid phase column (Discovery DSC18 1 g) and eluted with various concentrations of ethanol/water solutions. 50% and 75% ethanol elution from both water extracts showed anti-AR activity in 22RV1 cells using a prostate-specific antigen (PSA)-luciferase reporter assay (FIGS. 2A-2B). Based on the mass of two peaks observed in the LC-MS spectra of the ethanol elutions and Itokawa et al., Phytochemistry, 1989, 28, 3465-8, two major peaks in the 50% ethanol eluate were assigned as 1,3,6-Trihydroxy-2-methyl-9,10-anthracenedione 3-O-[α-L-Rhamnopyranosyl-(1→2)-β-D-glucopyranoside](RG) with MW 578.4, and 1,3,6-Trihydroxy-2-methyl-9,10-anthracenedione 3-O-[α-L-Rhamnopyranosyl-(1→2)-6-O-acetyl-β-D-glucopyranoside](RGA) with MW 620.4.

The separated anthraquinone moiety of RGA and RG was also found in the 75% eluate and assigned as 1,3,6-Trihydroxy-2-methyl-9,10-anthracenedione (TMT) with MW 270.2 (FIG. 2C).

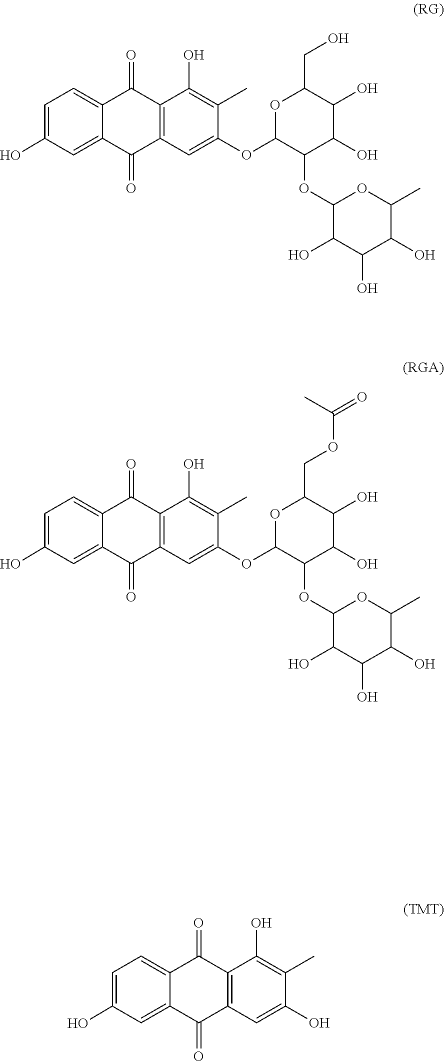 [SMILES: Cc1c(OC2OC(CO)C(O)C(O)C2OC2OC(C)C(O)C(O)C2O)cc2c(c1O)C(=O)c1ccc(O)cc1C2=O ; CC(=O)OCC1OC(Oc2cc3c(c(O)c2C)C(=O)c2ccc(O)cc2C3=O)C(OC2OC(C)C(O)C(O)C2O)C(O)C1O ; Cc1c(O)cc2c(c1O)C(=O)c1ccc(O)cc1C2=O]

### Example 2: Isolation and Positive Identification of RGA, RG and TMT

Large scale purification of RG and RGA from Rubia cordifolia extracts was then carried out. 2.5 g of Y1830 was dissolved in 10 ml HPLC grade water at 80 °C for 30 min. The solution of Y1830 was then centrifuged at 10000 rpm for 10 min and the supernatant was passed through a solid phase column (Discovery DSC18 10 g). The column was washed sequentially with 50 ml 10% EtOH and 50 ml 30% EtOH and finally 30 ml 50% EtOH was used to elute the fraction containing RG, RGA and TMT. The 50% EtOH fraction was vacuum dried and re-dissolved in 3 ml 50% EtOH before passing through a second solid phase column (Discovery DSC18 10 g). The second column was then washed with 30 ml 0.1% formic acid, 30 ml 10:90 [(Methanol:Acetonitrile 88:12):(0.1% formic acid)], 30 ml 30:70 [(Methanol:Acetonitrile 88:12):(0.1% formic acid)], and 30 ml 45:55 [(Methanol:Acetonitrile 88:12):(0.1% formic acid)]. RG containing fraction was collected by eluting with 30 ml 60:40 [(Methanol:Acetonitrile 88:12):(0.1% formic acid)]. An additional 25 ml 60:40 [(Methanol:Acetonitrile 88:12):(0.1% formic acid)] was eluted and discarded because it contained a mixture of both RG and RGA. The RGA containing fraction was then collected by eluting with 30 ml 75:25 [(Methanol:Acetonitrile 88:12):(0.1% formic acid)]. The fractions were then vacuum dried.

In order to produce enough TMT for testing, RG was hydrolyzed by adding 0.5 M HCl and stirring at 65 °C overnight. The reaction mixture was neutralized with NaOH and vacuum dried. The dried sample was dissolved in 3 ml 100% EtOH and passed through a solid phase column (Discovery DSC18 10 g). The column was washed with 30 ml 30:70 [(Methanol:Acetonitrile 88:12):(0.1% formic acid)], 30 ml 45:55 [(Methanol:Acetonitrile 88:12):(0.1% formic acid)] and 30 ml 75:25 [(Methanol:Acetonitrile 88:12):(0.1% formic acid)] to remove any residual RG. The fraction comprising TMT was eluted with 30 ml 100% (Methanol:Acetonitrile 88:12) and was vacuum dried. LC-MS (−ve scanning mode) was used to confirm the purity of the purified RG, RGA and TMT.

Since the acetyl group could alternatively be attached to the 3 or 4 position carbons of the β-D-glucopyranoside moiety of the RGA, both isomers having the same molecular weight as RGA in which the acetyl group is attached to the 6 position carbon of the β-D-glucopyranoside, NMR studies were undertaken to confirm the proper RGA structure. To confirm the final chemical structure of RGA an NMR-C600 was used for a ¹H scan and an NMR-C400 was used for a ¹³C scan.

¹H-NMR (DMSO-d₆) δ: 1.06 (3H, d, J=6 Hz, Rha-Me), 1.90 (3H, s, Ac-Me), 2.13 (3H, s, C-2-Me), 5.25 (1H, Rha-1H), 5.25 (1H, d, J=7.8 Hz, Rha-1H), 7.21 (1H, q, J=1.2, 8.4 Hz, H-7), 7.21 (1H, s, H-4), 7.45 (1H, d, J=2.4 Hz, H-5), 8.08 (1H, d, J=8.4 Hz, H-8), 13.20 (1H, s, C-6-OH) 13C-NMR (DMSO) δ: 186.86 (C-9), 182.12 (C-10), 170.78 (Ac—CO), 163.98 (C-1), 161.77 (C-6), 60.49 (C-3), 35.81 (C-4a), 132.35 (C-10a), 130.19 (C-8), 125.03 (C-8a), 121.95 (C-7), 121.00 (C-2), 113.04 (C-5), 111.08 (C-9a), 105.75 (C-4), 100.65 (C-1″), 97.74 (C-1′), 77.48 (C-3′), 76.74 (C-2′), 74.48 (C-5′), 72.42 (C-4″), 70.92 (C-3″), 70.75 (C-2″), 70.49 (C-4′), 69.00 (C-5″), 63.79 (C-6′), 20.86 (Ac-Me), 18.57 (C-6″), 9.20 (C-2-Me). The NMR spectra confirmed the final chemical structure for RGA as 1,3,6-Trihydroxy-2-methyl-9,10-anthracenedione 3-O-[α-L-Rhamnopyranosyl-(1→2)-6-O-acetyl-β-D-glucopyranoside].

Additionally, it was found that treatment of RGA (1,3,6-Trihydroxy-2-methyl-9,10-anthracenedione 3-O-[α-L-Rhamnopyranosyl-(1→2)-6-O-acetyl-β-D-glucopyranoside]) with 0.2 N NaOH in a 50% methanol solution for 10 min could remove the acetyl group of RGA, yielding RG (FIG. 2D).

Quantification of RG, RGA and TMT content in extracts of Y9, Y1830 and Y1831 was done by comparing the area of peaks of the purified RG, RGA and TMT to the area of peaks of water extract of Y9, Y1830 and Y1831 using LC-MS (Table 2). The amount of RGA in herbal each extract had some correlation to the anti-AR activity of the herbal water extracts. Y1830 extracts were found to contain more RGA than Y9 and Y1831 extracts and the Y1830 extracts demonstrated the most anti-AR activity (FIG. 1).

TABLE 2
Quantification of RG, RGA and TMT in Y9, Y1830 and Y1831.

| | Contained . . . | | |
|---|---|---|---|
| Each mg of . . . | RG (μg) | RGA (μg) | TMT (μg) |
| Y9 | 4.20 | 3.2 | 1.6 |
| Y1830 | 4.10 | 4.4 | 1.2 |
| Y1831 | 0.25 | 1.2 | 0.0 |

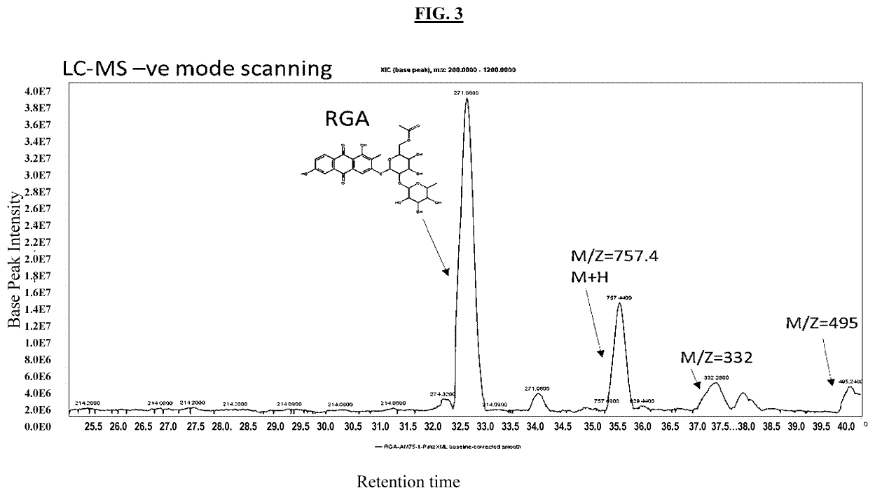

When positive scanning mode was used in LC-MS detection, in addition to RGA, a few more compounds could be detected with M/Z=757.4 (M+H), M/Z=332, M/Z=495 (FIG. 3) that could also be potentially active compounds in RGA containing fraction.

### Example 3: Anti-AR Activity of the Isolated Compounds

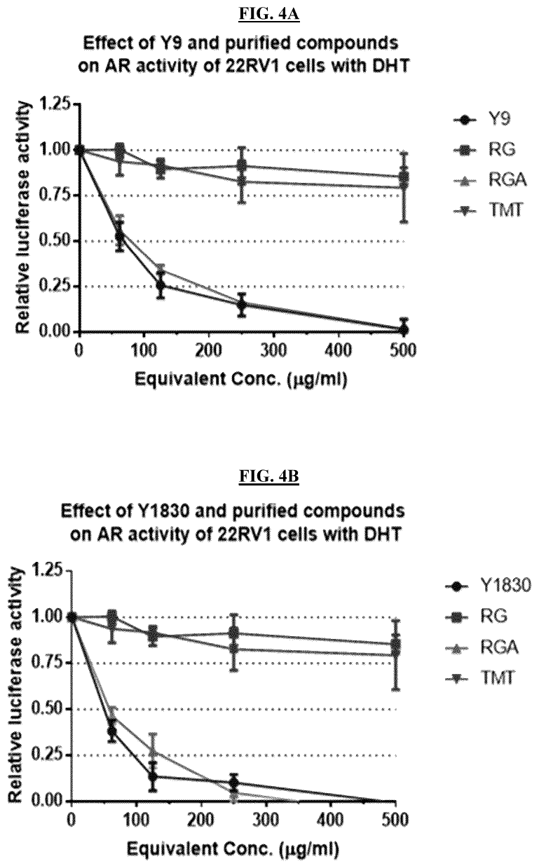

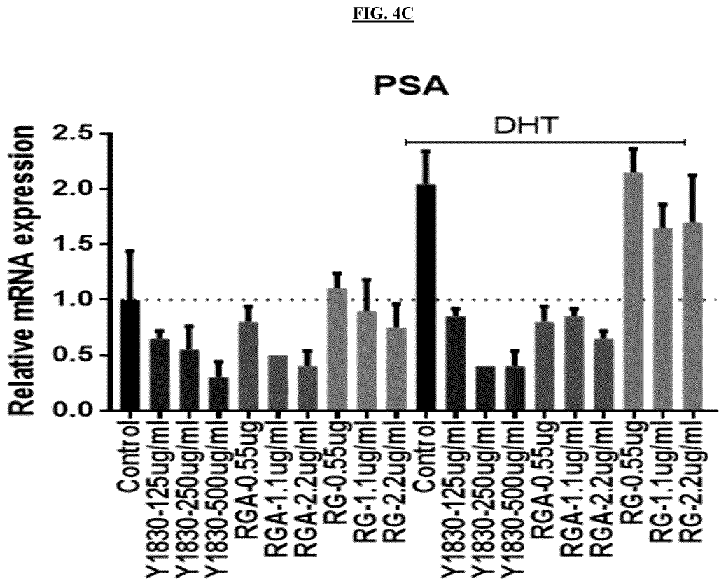

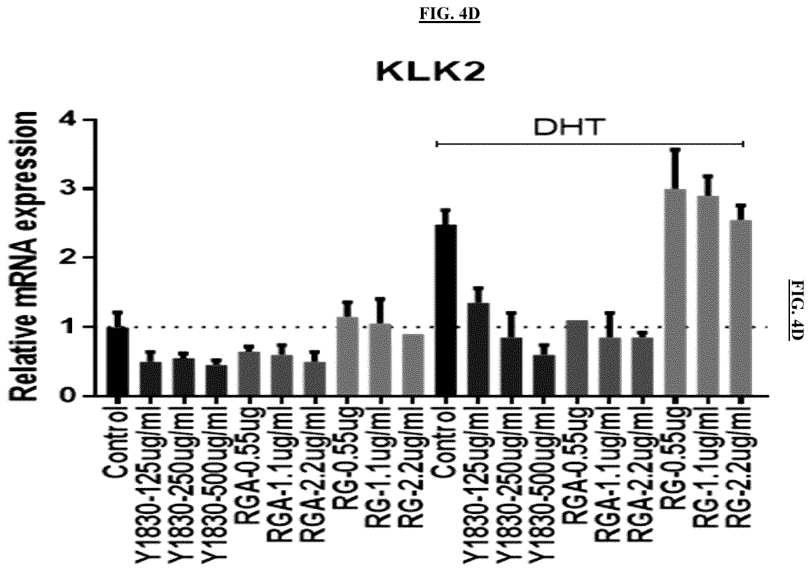

Equivalent doses of Y9, Y1830, RG (or the fraction comprising RG), RGA (or the fraction comprising RGA) and TMT (or the fraction comprising TMT) were tested for their inhibitory effect on AR mediated transcriptional response in 22RV1 cells in the presence of DHT (FIGS. 4A-4B). Results indicated that RGA (or the fraction comprising RGA) demonstrated very similar anti-AR activity as the Y1830 water extract while RG (or the fraction comprising RG) and TMT (TMT containing fraction) showed very weak activity against AR. qPCR results showed that Y1830 and RGA (or the fraction comprising RGA) had similar activity in inhibiting the mRNA expression of endogenous AR target genes PSA and KLK2 (FIGS. 4C-4D). RG (or the fraction comprising RG) did not demonstrate this level of activity. These results confirmed that RGA (or the fraction comprising RGA) plays a key role in the AR inhibitory activity of Rubia cordifolia.

### Example 4: Treatment of Prostate Cancer Using Extracts and Isolated Compounds

Based on the prior results, it was hypothesized that Rubia cordifolia and RGA have potential applications for the treatment of prostate hyperplasia and prostate cancer. Because the 22RV1 cells that were tested were castration-resistant prostate cancer cells that show enzalutamide resistance, Rubia cordifolia and RGA could be useful for treating castration-resistant prostate cancer cells that have shown enzalutamide resistance.

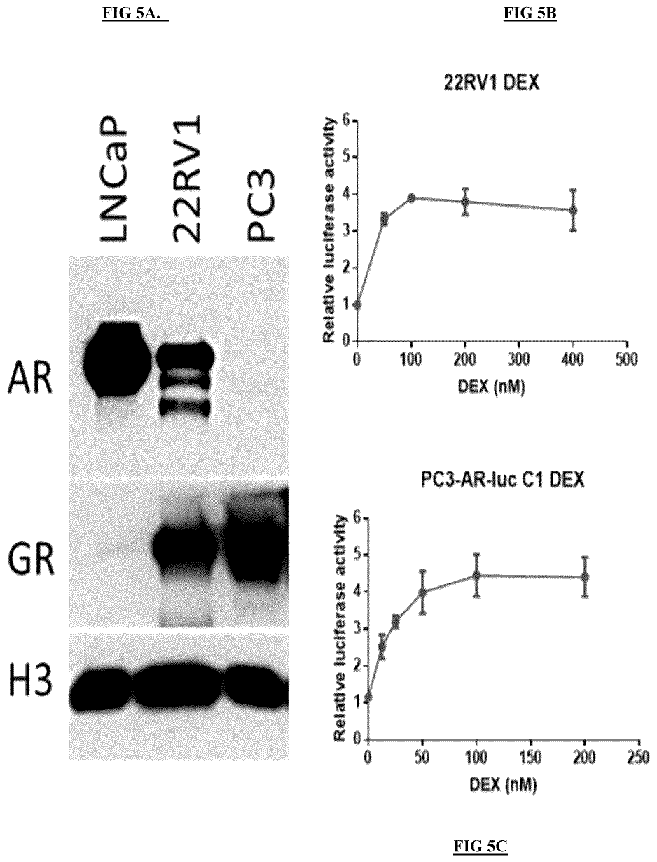

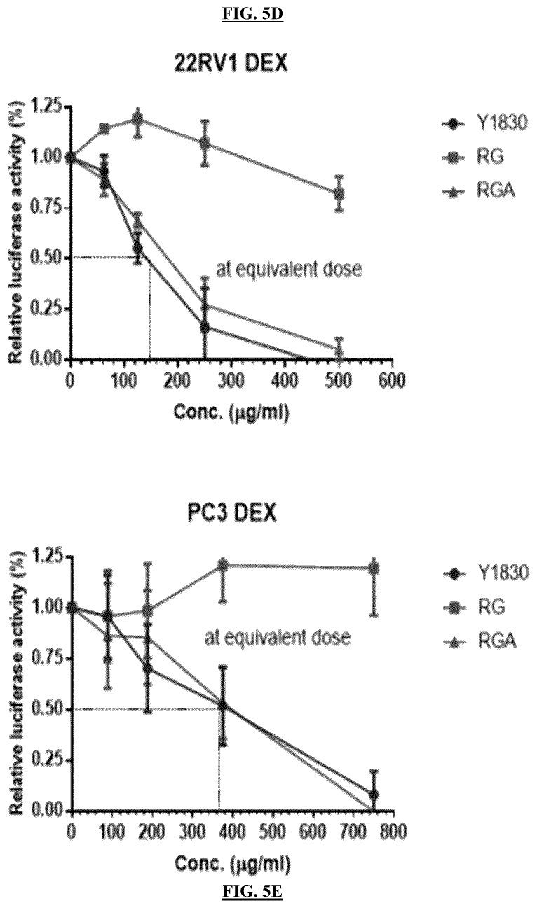

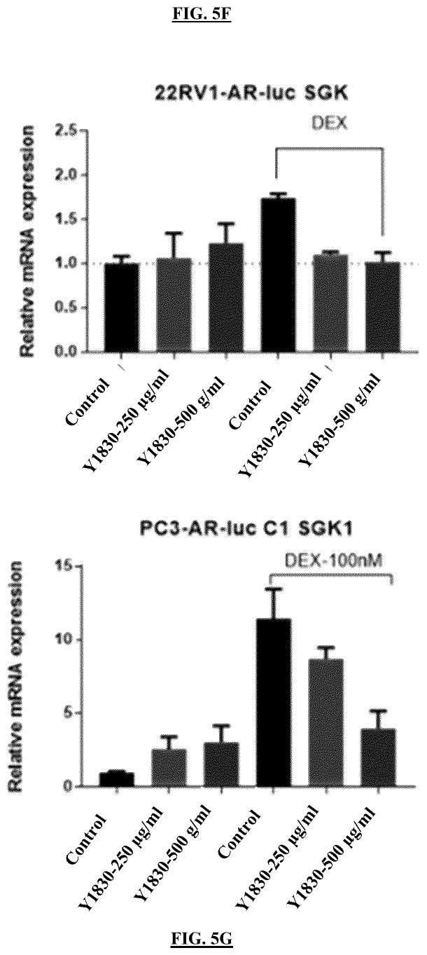

About 30% of enzalutamide resistance patients have shown overexpression of glucocorticoid receptor (GR). GR can replace AR function and drive tumor growth in enzalutamide resistance prostate cancer. Y1830 water extract and RGA (or the fraction comprising RGA) were found to have an inhibitory effect on dexamethasone (DEX) induced transcriptional response in 22RV1 and PC3 PSA-luciferase reporter cells (FIGS. 5A-5E). Y1830 water extract and RGA (or the fraction comprising RGA) also inhibited DEX induced SGK1 mRNA expression in 22RV1 or PC3 cells. Results showed that Y1830 and RGA (or the fraction comprising RGA) could be potential treatments for GR overexpression mediated enzalutamide resistance prostate cancer.

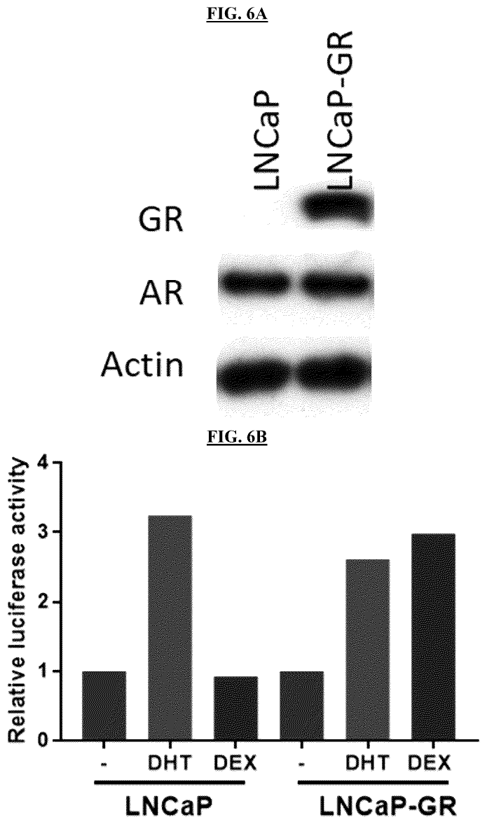

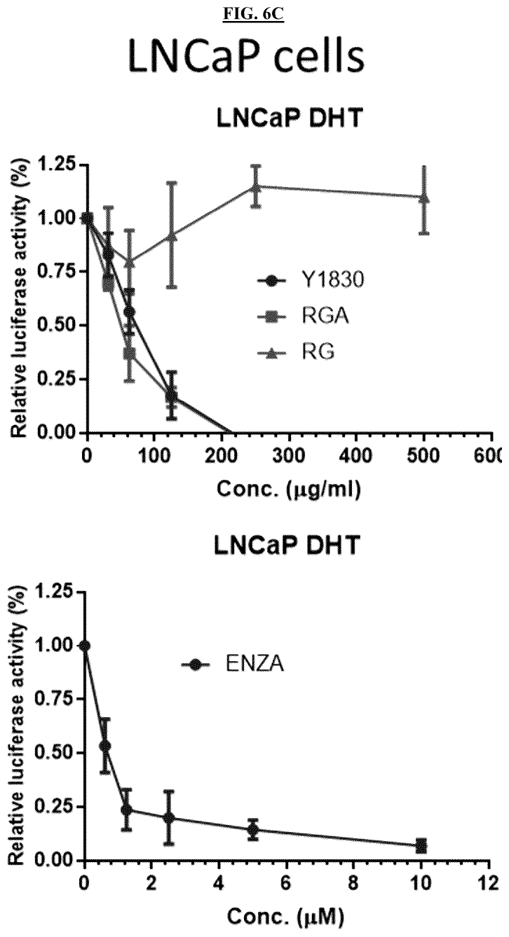

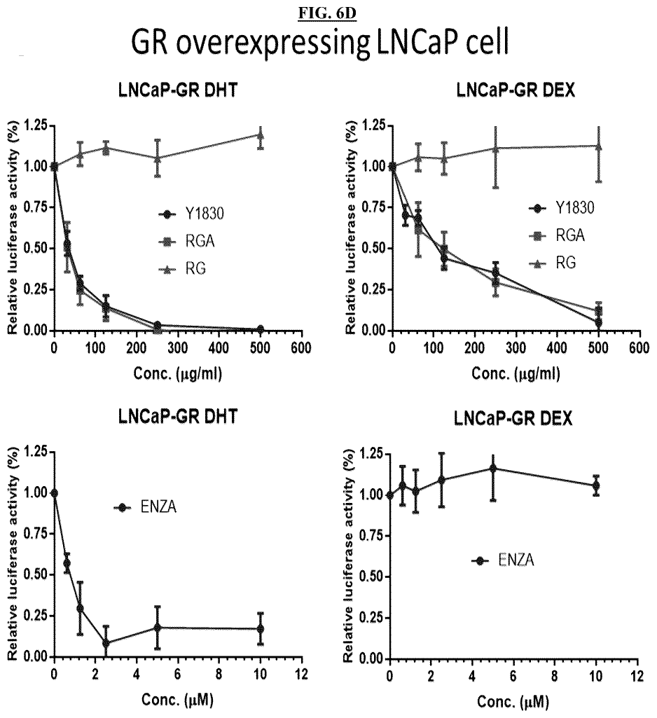

When glucocorticoid receptor (GR) was overexpressed in LNCaP cells (FIG. 6A), LNCaP-GR cells became responsive to DEX stimulation as reflected by luciferase activity (FIG. 6B). When comparing enzalutamide treatment with Y1830 or RGA (or the fraction comprising RGA) treatment, enzalutamide could only inhibit DHT induced transcriptional response but not DEX induced transcriptional response (FIG. 6C). Y1830 water extract and RGA (or the fraction comprising RGA) could effectively inhibit DHT or DEX induced transcriptional response (FIG. 6D).

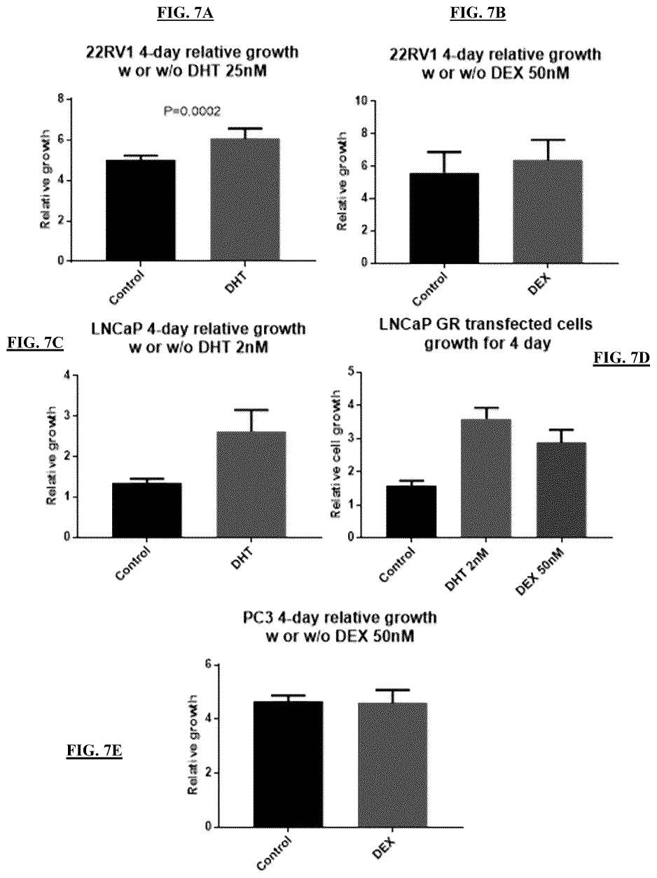

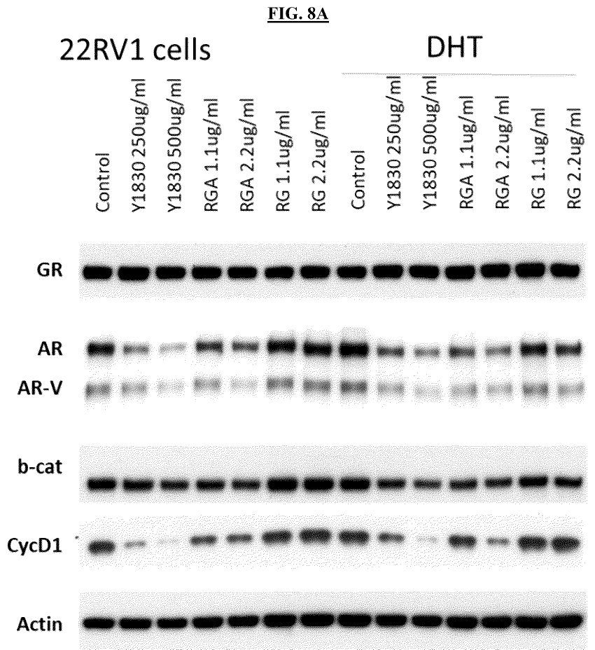

Enzalutamide, Y1830, RGA (or the fraction comprising RGA), and RG (or the fraction comprising RG) were further tested for their ability to inhibit the growth of different prostate cell lines in the presence or absence of DHT or DEX. The growth of 22RV1 cells were found to be slightly dependent on DHT but not DEX (FIGS. 7A-7B). As expected 22RV1 were found to be resistant to enzalutamide treatment. Y1830 and RGA (or the fraction comprising RGA) showed similar growth inhibitory effect on 22RV1 cells in the presence or absence of DHT and DEX (Table 3). The results indicate that 22RV1 cell growth was more susceptible to Y1830 and RGA (or the fraction comprising RGA) in the presence of DHT or DEX. In the absence of DHT, LNCaP cells were found to not be susceptible to enzalutamide, Y1830 or RGA (or the fraction comprising RGA) treatment. However, DHT was found to stimulate LNCaP cell growth (FIG. 7C). In the presence of DHT, LNCaP cells were more sensitive to enzalutamide, Y1830 and RGA (or the fraction comprising RGA) treatment (Table 3). DEX was found to stimulate GR overexpressing LNCaP (LNCaP-GR) cell growth and made LNCaP cells resistant to enzalutamide. Y1830 and RGA (or the fraction comprising RGA) effectively inhibited LNCaP-GR cell growth in the presence of either DHT or DEX. Y1830 and RGA also inhibited PC3 cell growth, but less effectively in the presence of DEX. Under all conditions, RG (or the fraction comprising RG) did not any show inhibitory effect on cell growth up to 500 μg/ml. In summary, Y1830 and RGA (or the fraction comprising RGA) showed similar cell growth inhibition but potency was affected by cell type and the presence or absence of DHT or DEX. These results suggest that Y1830 and RGA (or the fraction comprising RGA) may be useful for treating androgen dependent and androgen independent prostate cancer. Additionally, Y1830 and RGA (or the fraction comprising RGA) may be useful for treating enzalutamide resistant cancers, as exemplified in the 22RV1 cell assays. Y1830 and RGA (or the fraction comprising RGA) may also be useful for inhibiting GR mediated enzalutamide resistance as exemplified in LNCaP-GR DEX conditions.

TABLE 3
IC₅₀ of various compounds for the inhibition
of growth of different prostate cell lines.

| | | | RGA (or | RG (or |
|---|---|---|---|---|
| | | | the fraction | the fraction |
| | Enzalutamide | Y830 | comprising | comprising |
| | (μM) | (μg/mL) | RGA) | RG) |
| 22Rv1 | >5 | 306 | 306 | >500 |
| 22Rv1 w/DHT | >5 | 253 | 253 | >500 |
| 22Rv1 w/DEX | >5 | 220 | 220 | >500 |
| LNCaP | >5 | >500 | >500 | >500 |
| LNCaP w/DHT | 0.5 | 50 | 50 | >500 |
| LNCaP-GR | >5 | 250 | 220 | >500 |
| LNCaP-GR | 0.5 | 40 | 40 | >500 |
| w/DHT | | | | |
| LNCaP-GR | >5 | 60 | 60 | >500 |
| w/DEX | | | | |
| PC3 | >5 | 146 | 146 | >500 |
| PC3 w/DEX | >5 | 293 | 320 | >500 |

IC₅₀ for RGA and RG was standardized to equivalent concentration of Y1830. Y1830 1 mg/ml was found to contain 4.4 μg/mL of RGA.
DHT: 25 nM was added to 22RV1 cells. 2 nM was added to LNCaP cells or LNCaP-GR (GR overexpressing) cells.
DEX (dexamethasone): 50 nM was added to the noted cells.
Cell growth was determined after 4 days of incubation. IC₅₀ was determined as the concentration required to inhibit 50% of cell growth.

### Example 5: Y1830 and RGA/the Fraction Comprising RGA Effect on Hormone Receptor Protein Expression

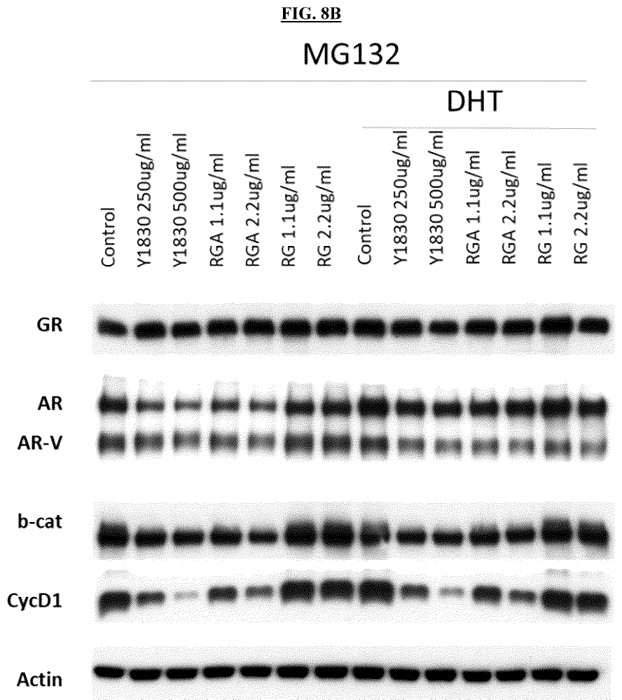

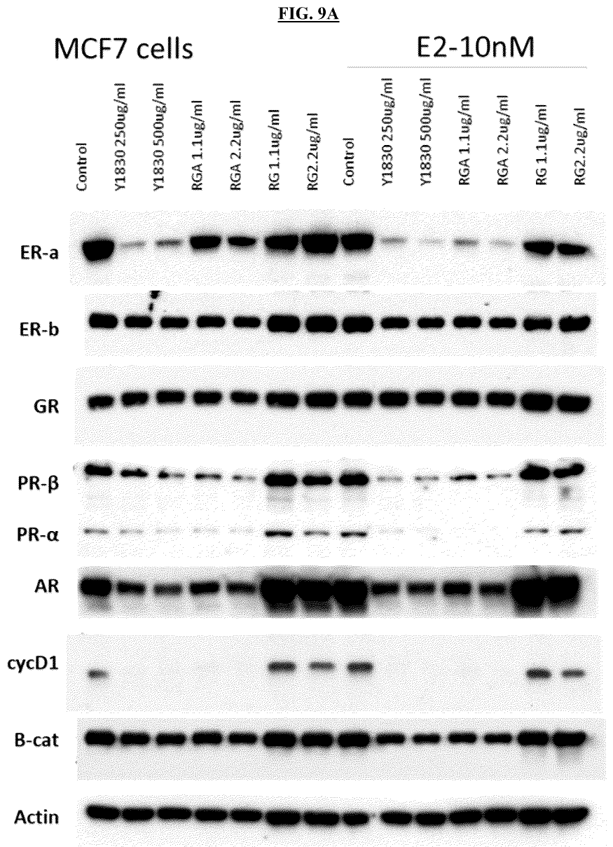

In the presence or absence of DHT, Y1830 and RGA (or the fraction comprising RGA) were found to downregulate AR and AR-V (truncated AR) but not GR (FIG. 8A) in 22RV1 cells. The downregulation of AR protein by Y1830 and RGA could be counteracted by the addition of MG132 (a proteasome inhibitor) (FIG. 8B). Without intending to be limited to any particular theory, this result suggests that the downregulation of AR protein by Y1830 and RGA (or the fraction comprising RGA) could involve the activation of proteasome pathway(s). It was also found that Y1830 and RGA (or the fraction comprising RGA) inhibited GR activity without downregulating GR protein expression.

In the presence or absence of DHT, Y1830 and RGA (or the fraction comprising RGA) had a strong inhibitory effect on cyclin D1 (cycD1) protein expression and moderate inhibitory effect on beta-catenin (b-cat) protein expression (FIG. 8A). However, the downregulation of cycD1 and b-cat protein by Y1830 and RGA was not affected by addition of MG132. This result suggests that activation of proteasome pathway(s) was not the mechanism of action of the downregulation of cycD1 and b-cat by Y1830 and RGA (or the fraction comprising RGA). RG (or the fraction comprising RG) did not have impact on the protein expression of GR, AR, AR-V, b-cat, or cycD1 (FIGS. 8A-8B).

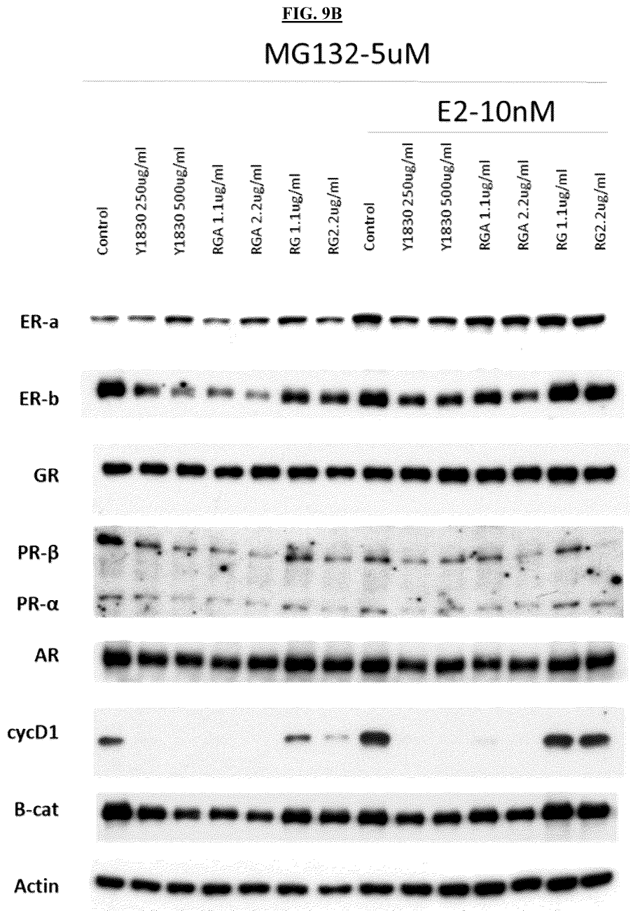

In the presence or absence of 17β-Estradiol (E2), Y1830 and RGA (or the fraction comprising RGA) were found to downregulate estrogen receptor ERa, AR, progesterone receptor PR (a and b) and slightly downregulate estrogen receptor ERb in MCF7 breast cancer cells (FIG. 9A). The downregulation of ERa and AR proteins was counteracted by the addition of MG132. However, downregulation of PR was unaffected by MG132 (FIG. 9B). These results suggest the involvement of proteasome pathway(s) for AR and ERa downregulation but not ERb and PR. Again, Y1830 and RGA (or the fraction comprising RGA) did not inhibited GR protein expression of MCF7 cells, similar to the results observed in 22RV1 cells.

In the presence or absence of E2, Y1830 and RGA (or the fraction comprising RGA) had a strong inhibitory effects on cyclin D1 (cycD1) protein expression and a moderate inhibitory effect on beta-catenin (b-cat) expression (FIG. 9A). However, the downregulation of cycD1 or b-cat protein by Y1830 and RGA (or the fraction comprising RGA) was not affected by MG132. This result suggests that activation of proteasome pathway(s) was not the mechanism of action of the downregulation of cycD1 and b-cat by Y1830 and RGA in the MCF7 cells. RG (or the fraction comprising RG) did not have any impact on the protein expression of all protein examined.

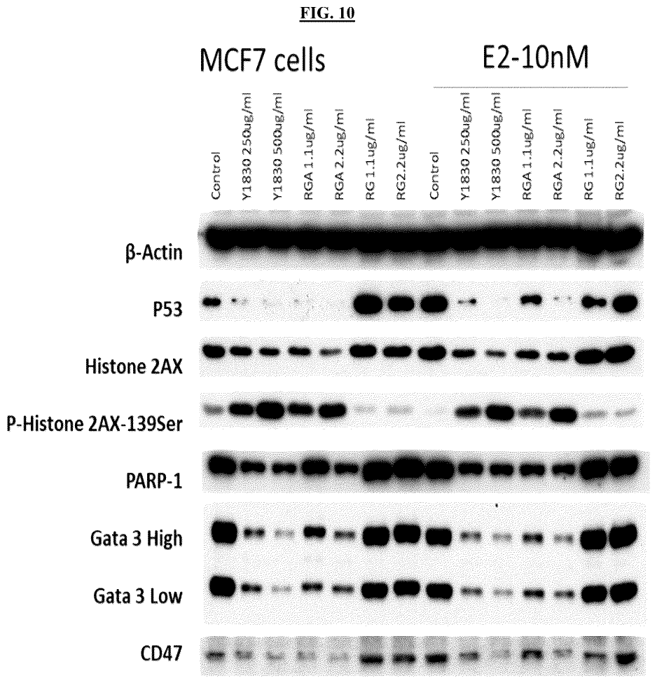

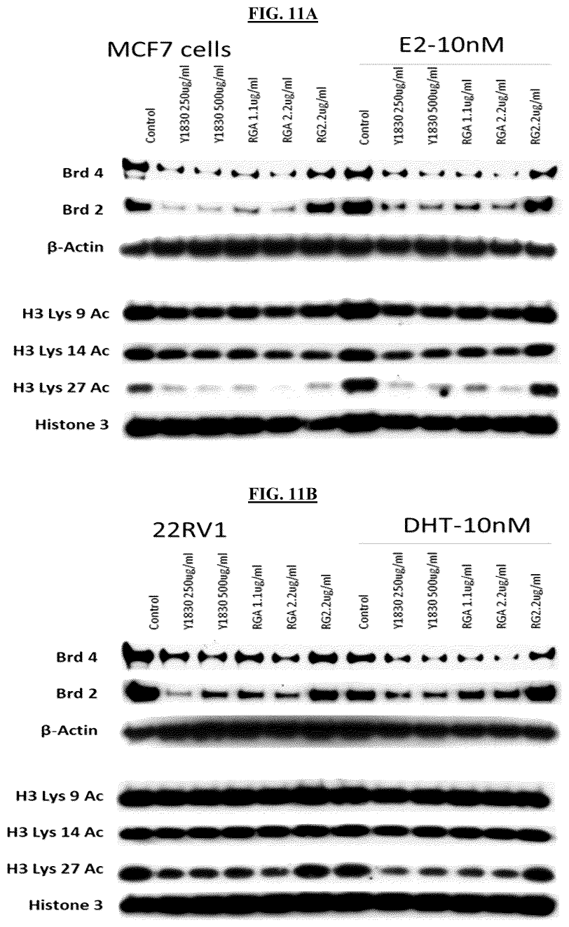

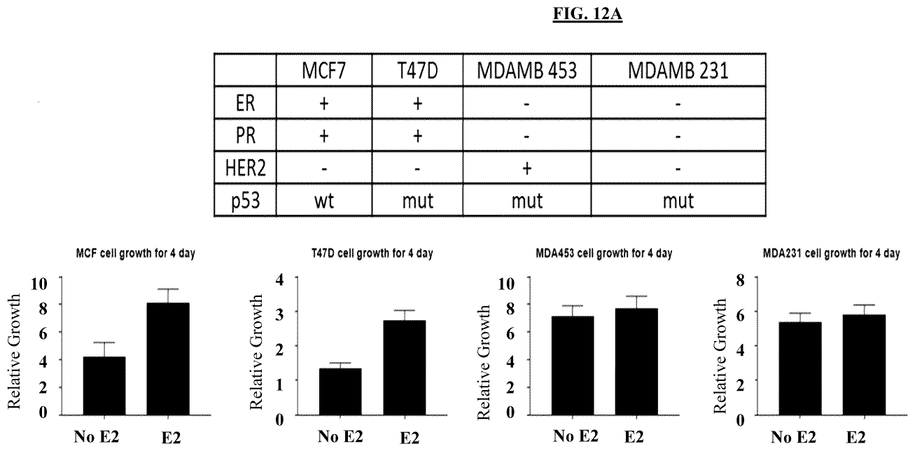

In MCF7 cells, in presence or absence of E2, Y1830 and RGA (or the fraction comprising RGA) triggered histone 2AX serine 139 phosphorylation, which is a marker for DNA double-stranded breakage. Y1830 and RGA (or the fraction comprising RGA) were also found to downregulate Poly[ADP-ribose]polymerase-1 (PARP-1) protein which has an important role in the repair of single-stranded DNA (ssDNA) breaks. In addition, Y1830 and RGA downregulated tumor protein p53 in wt MCF7 cells (FIG. 10). These results suggest that Y1830 and RGA (or the fraction comprising RGA) might hinder the DNA repair process. Gata3 is known to operate in a positive feedback loop with ERa, therefore the observed simultaneous downregulation of ERa protein and Gata3 protein by Y1830 and RGA (or the fraction comprising RGA) was expected. Downregulation of CD47 (on tumor cells) promotes phagocytosis of tumor cells by macrophages by preventing inhibition of signal regulatory protein-α (SIRPa) on the macrophages. Therefore, it is possible that downregulation of CD47 by Y1830 and RGA (or the fraction comprising RGA) could be useful in immunotherapy applications (FIG. 10).

The BET (Bromodomain and Extra-Terminal Domain) subfamily of bromodomain proteins (Brd2, Brd3, Brd4) are important for both ERα and AR dependent enhancer activation and gene transcription. BET inhibitors have been shown to inhibit breast cancer cell growth, including triple negative breast cancer (TNBC). Y1830 and RGA (or the fraction comprising RGA) decreased Brd2 and Brd4 protein expression of MCF7 (with or without E2) or 22RV1 (with or without DHT) (FIGS. 11A-11B). Y1830 and RGA (or the fraction comprising RGA) selectively decreased H3K27 acetylation in MCF7 and 22RV1 cells (FIGS. 11A-11B). Y1830 and RGA (or the fraction comprising RGA) did not have a large impact on H3K9 acetylation or H3K14 acetylation (FIGS. 11A-11B). These results suggest that Y1830 and RGA (or the fraction comprising RGA) could reduce binding of Brd proteins to promoter/enhancer regions of ERα and AR target genes and therefore affect their transcription. These results also suggest that Y1830 and RGA (or the fraction comprising RGA) are not pan-inhibitors for histone acetyltransferases (HAT). Y1830 and RGA (or the fraction comprising RGA) were only found to inhibit transcription of a subset of genes which are dependent on H3K27Ac.

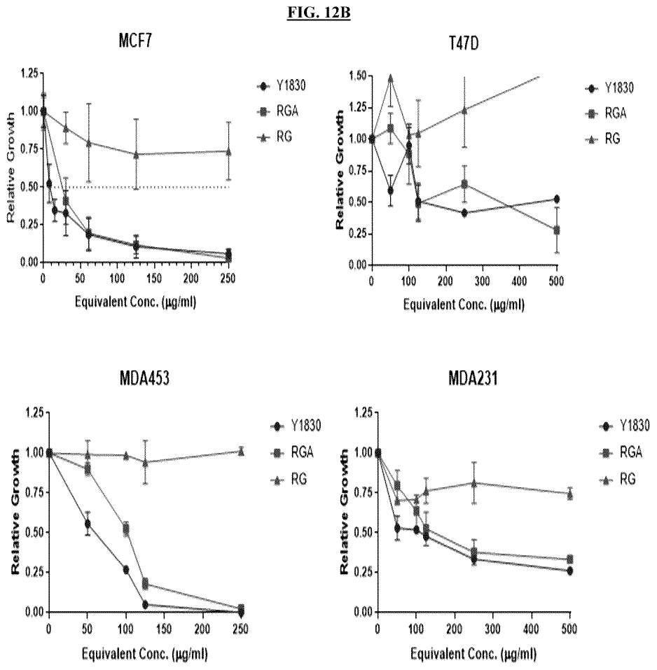

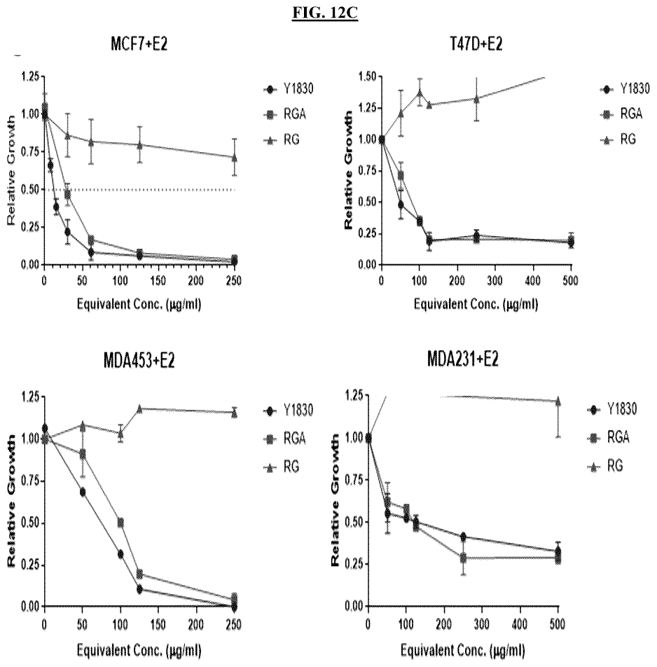

The effects of Y1830, RGA (or the fraction comprising RGA) and RG (or the fraction comprising RG) on the growth of MCF7, T4D, MDAMB453 and MDAMBA231 breast cancer cells, in the presence or absence of E2 was measured for 4 days. Results indicated that T4D cell growth was almost totally dependent on E2 (FIG. 12A). Although cell growth of MCF7 cells was not absolutely depend on E2, addition of E2 (10 nM) did speed up the growth of MCF7 cells one-fold (FIG. 12A). MDAMB453 and MDAMBA231 cell growth were independent of E2 (FIG. 12A). Y1830 and RGA (or the fraction comprising RGA) showed similar inhibition on the growth of MCF7 cells in the presence or absence of E2 while RG had very weak growth inhibition on MCF7 cells (FIGS. 12B-12C). When T4D cells were under E2 stimulation, Y1830 and RGA (or the fraction comprising RGA) showed a strong inhibitory effect on T4D cell growth (FIG. 12C). Y1830 and RGA (or the fraction comprising RGA) also inhibited the growth of double negative (ER−ve. PR−ve) MDA-MB-453 cells and triple negative (ER−ve, PR−ve, HER2−ve) MDAMB-231 cells (FIGS. 12B-12C).

### Example 6: Indoleamine-Pyrrole 2,3-Dioxygenase (IDO) Inhibition by Y1830 and RGA

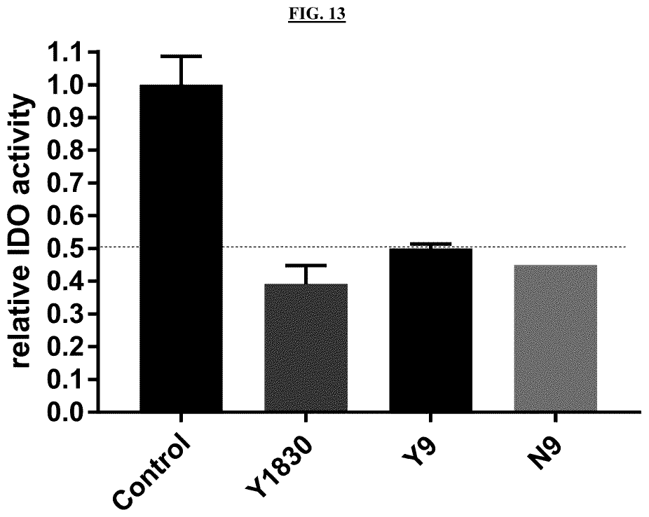

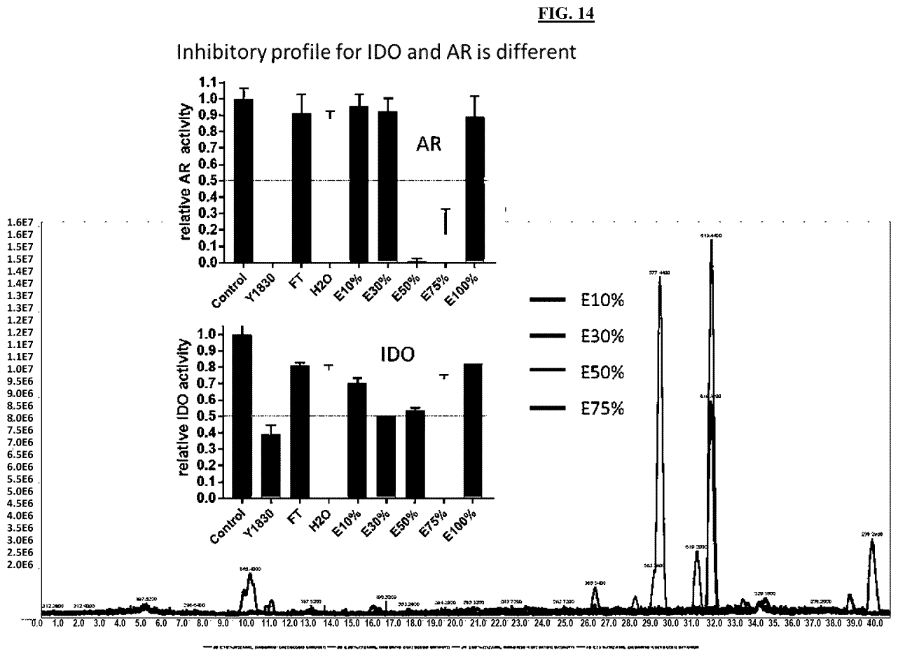

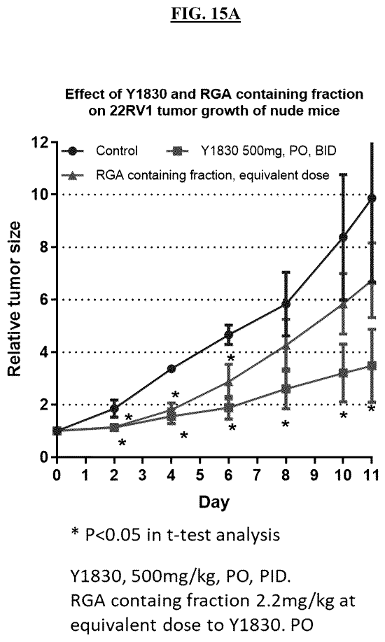

Indoleamine-pyrrole 2,3-dioxygenase (IDO) is the enzyme responsible for metabolizing L-tryptophan, converting it into kynurenine. IDO could a key role in resistance to anti-PD1 and anti-CTLA4 therapies. IDO inhibitors enhance the action of anti-PD1, anti-PD-L1, anti-CTLA4 therapies in different types of tumors in animals. IDO inhibitory activity was tested for different preparations of Rubia cordifolia (Y1830, Y9, N9) at 500 μg/ml. The extracts showed an inhibitory effect of about 50% on IDO activity in in vitro assays (FIG. 13). Inhibition profiles of different ethanol elutions of Y1830 on AR activity and IDO activity were compared (FIG. 14). For IDO, 30% EtOH and 50% EtOH elutions of Y1830 showed substantial inhibition. For AR, the 30% EtOH elution had no activity while the 50% EtOH and 75% EtOH elutions showed inhibition. The 30% EtOH had selective activity against IDO, suggesting that at least one compound present in the 30% EtOH fraction, and not present in the 50% EtOH and 75% EtOH elutions was responsible for this activity. A mass peak with a MW of 366.04 at 26 min was found to be unique for the 30% EtOH fraction (FIG. 14).

### Example 7: Y1830 and RGA (or the Fraction Comprising RGA) Inhibits 22RV1 Tumor Growth in Nude Mice

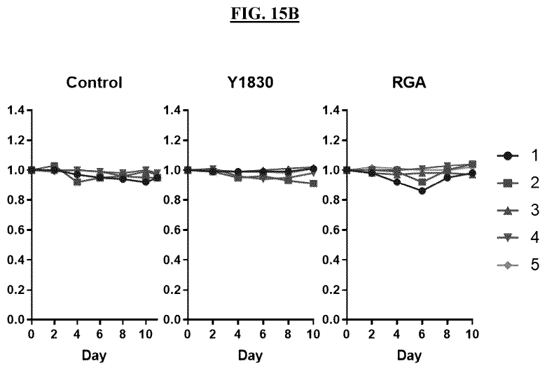

22RV1 cells (enzalutamide resistant prostate cancer cells) were subcutaneously implanted into male nude mice. When tumors reached 5 mm×5 mm, Y1830 water extract (500 mg/kg, BID, PO) and RGA (or the fraction comprising RGA) at equivalent dose to Y1830 were used to treat animals for 11 days. As shown in FIG. 15A, Y1830 showed significant inhibition on 22RV1 tumor growth from day 2 to day 11 (P<0.05). RGA (or the fraction comprising RGA) showed significant inhibition on 22RV1 tumor growth from day 2 to day 6 (P<0.05) (FIG. 15A). Both treatments did not cause animal body weight loss implying no significant overall toxicity to animals (FIG. 15B). In conclusion, Y1830 had stronger anti-tumor activity than the fraction comprising RGA against 22RV1 tumor growth. Therefore, it is further concluded that compounds other than molecules in RGA containing fraction of Y1830 total extract may also have anti-tumor activity or prevent tumor to develop drug resistant.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating at least one disease or disorder in a subject in need thereof, the method comprising administering to the subject a therapeutically effective amount of an herbal extract of Rubia cordifolia or a fraction thereof, or any active chemical species present in the herbal extract or the fraction thereof, wherein the disease or disorder is related to the activity of at least one steroid hormone receptor or the expression of at least one protein selected from the group consisting of Brd4, Brd2, cyclin D1, p53, Gata3, Poly[ADP-ribose]polymerase-1 (PARP-1) and CD47.

2. The method of claim 1, wherein the active chemical species present in the herbal extract or the fraction thereof is selected from the group consisting of 1,3,6-Trihydroxy-2-methyl-9,10-anthracenedione 3-O-[α-L-Rhamnopyranosyl-(1→2)-6-O-acetyl-β-D-glucopyranoside](RGA), 3,6-Trihydroxy-2-methyl-9,10-anthracenedione 3-O-[α-L-Rhamnopyranosyl-(1→2)-β-D-glucopyranoside](RG) and salts, solvates, isomers, tautomers or prodrugs thereof

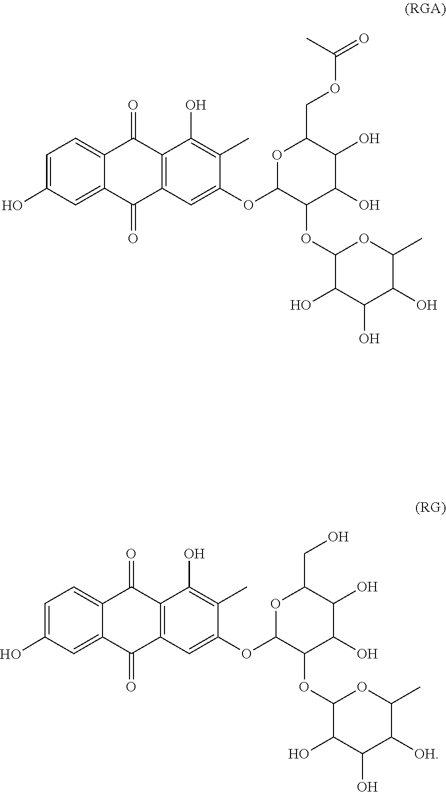 [SMILES: CC(=O)OCC1OC(Oc2cc3c(c(O)c2C)C(=O)c2ccc(O)cc2C3=O)C(OC2OC(C)C(O)C(O)C2O)C(O)C1O ; Cc1c(OC2OC(CO)C(O)C(O)C2OC2OC(C)C(O)C(O)C2O)cc2c(c1O)C(=O)c1ccc(O)cc1C2=O]

3. The method of claim 1, wherein the at least one steroid hormone receptor is at least one selected from the group consisting of progesterone receptors, estrogen receptors, androgen receptors, and glucocorticoid receptors.

4. The method of claim 1, wherein the at least one disease or disorder related to the activity of at least one steroid hormone receptor is a cancer selected from the group consisting of prostate cancer, breast cancer, ovarian cancer, lung cancer, uterine cancer, pancreatic cancer, colon cancer, hepatocellular carcinoma, glioblastoma, multiple myeloma, NUT carcinoma, leukemia, and lymphoma.

5. The method of claim 4, wherein the cancer is a treatment resistant cancer selected from the group consisting of castration resistant prostate cancer, enzalutamide resistant prostate cancer, glucocorticoid receptor mediated resistant prostate cancer, BRCA1 (double strand break repair) deficiency cancer, double negative breast cancer, and triple negative breast cancer.

6. The method of claim 1, wherein the at least one disease or disorder related to the activity of at least one steroid hormone receptor is selected from the group consisting of prostate hyperplasia, Cushing's syndrome, androgenetic alopecia, acne, seborrhea, hirsutism (excessive body hair), hidradenitis suppurativa, sexual dysfunction, precocious puberty (in both males and females), polycystic ovary syndrome, mastodynia (breast pain/tenderness), breast fibroids, mammoplasia (breast enlargement), macromastia (breast hypertrophy), gynecomastia, melasma, menorrhagia, endometriosis, endometrial hyperplasia, adenomyosis, uterine fibroids, and posttraumatic stress disorder (PTSD).

7. The method of claim 1, wherein the at least one disease or disorder related to expression of the at least one protein is selected from the group consisting of Huntington's disease, schizophrenia, psoriasis, mantle cell lymphoma, breast carcinoma, bladder cancer, pituitary adenomas, parathyroid adenoma, pancreatic carcinoma, head and neck squamous cell carcinomas, and non-small cell lung cancers.

8. The method of claim 1, wherein the method downregulates the expression of at least one protein selected from the group consisting of Brd4, Brd2, cyclin D1, p53, Gata3, Poly[ADP-ribose]polymerase-1 (PARP-1), and CD47.

9. The method of claim 1, wherein the administration inhibits hyperacetylation of histone H3 in the subject.

10. The method of claim 1, wherein the administration promotes the phagocytosis of cancerous tumor cells in the subject.

11. The method of claim 1, wherein the administration inhibits indoleamine-pyrrole 2,3-dioxygenase (IDO) activity in the subject.

12. The method of claim 1, wherein the subject is further administered at least one immune checkpoint inhibitor.

13. The method of claim 12, wherein the at least one immune checkpoint inhibitor is selected from the group consisting of an anti-PD1, an anti-PD-L1, and an anti-CTLA4.

14. The method of claim 12, wherein the at least one immune checkpoint inhibitor is selected from the group consisting of Ipilimumab, Avelumab, Pembrolizumab, Nivolumab, Durvalumab, and Atezolizumab.

15. The method of claim 1, wherein the subject is a mammal.

16. The method of claim 15, wherein the subject is human.


SEQUENCE LISTING (8 sequences)

SEQ ID NO: 1 - DNA, 21 bp, synthetic construct
  misc_feature 1..21: note = Primer
  source 1..21: mol_type = other DNA
cctggcttccgcaacttacac

SEQ ID NO: 2 - DNA, 22 bp, synthetic construct
  misc_feature 1..22: note = Primer
  source 1..22: mol_type = other DNA
ggacttgtgcatgcggtactca

SEQ ID NO: 3 - DNA, 23 bp, synthetic construct
  misc_feature 1..23: note = Primer
  source 1..23: mol_type = other DNA
ggtggctgtgtacagtcatggat

SEQ ID NO: 4 - DNA, 23 bp, synthetic construct
  misc_feature 1..23: note = Primer
  source 1..23: mol_type = other DNA
tgtcttcaggctcaaacaggttg

SEQ ID NO: 5 - DNA, 24 bp, synthetic construct
  misc_feature 1..24: note = Primer
  source 1..24: mol_type = other DNA
accagaggagttcttgaccccaaa

SEQ ID NO: 6 - DNA, 20 bp, synthetic construct
  misc_feature 1..20: note = Primer
  source 1..20: mol_type = other DNA
ccccagaatcacccgagcag

SEQ ID NO: 7 - DNA, 21 bp, synthetic construct
  misc_feature 1..21: note = Primer
  source 1..21: mol_type = other DNA
gccacggctgcttccagctcc

SEQ ID NO: 8 - DNA, 24 bp, synthetic construct
  misc_feature 1..24: note = Primer
  source 1..24: mol_type = other DNA
ttgtgctgggtgccagggcagtga